(19) United States
(12) Patent Application Publication
(10) Pub. No.: US 2023/0044570 A1
(43) Pub. Date: Feb. 9, 2023

(54) METHOD FOR DETERMINING A MEASURE CORRELATED TO THE PROBABILITY THAT TWO MUTATED SEQUENCE READS DERIVE FROM THE SAME SEQUENCE COMPRISING MUTATIONS

(71) Applicant: Illumina Singapore Pte. Ltd, Northtech (SG)

(72) Inventor: Aaron Earl Darling, Sydney (AU)

(21) Appl. No.: 17/754,303

(22) PCT Filed: Sep. 29, 2020
(86) PCT No.: PCT/GB2020/052358

§ 371 Date: Mar. 29, 2022

(30) Foreign Application Priority Data
Sep. 30, 2019 (GB) 1914064.9

(51) Int. Cl. G16B 30/10; C12Q 1/6869; C12Q 1/6827; G16B 30/20; G16B 40/00

(52) U.S. Cl. CPC G16B 30/10; C12Q 1/6869; C12Q 1/6827; G16B 30/20; G16B 40/00

(57) ABSTRACT

Disclosed is a computer-implemented method for determining a measure correlated to the probability that two mutated sequence reads derive from the same sequence comprising mutations. The method comprises receiving mutated sequence reads each corresponding to a subsequence of a sequence comprising mutations compared to a sequence not comprising mutations, applying a common minimizer function to each mutated sequence read, to determining minimizers for each mutated sequence read, determining positions of the one or more minimizers in each mutated sequence read, determining positions of mutations in each mutated sequence read, and for at least two mutated sequence reads with a common minimizer, counting the number of mutations with matching position and/or mismatching position when the respective minimizers are aligned. Also disclosed is a corresponding method for determining at least a portion of a sequence of at least one target template nucleic acid molecule.

### FIELD OF THE INVENTION

The invention relates to a computer-implemented method for determining a measure correlated to the probability that two mutated sequence reads derive from the same sequence comprising mutations, a method for generating at least a portion of a sequence, and a method for determining a sequence of at least one target template nucleic acid molecule.

### BACKGROUND OF THE INVENTION

The ability to sequence nucleic acid molecules is a tool that is very useful in a myriad of different applications. However, it can be difficult to determine accurate sequences for nucleic acid molecules that comprise problematic structures, such as nucleic acid molecules that comprise repeat regions. It can also be difficult to resolve structural features, such as the haplotype structure of diploid and polyploid organisms and structural variants in the genomes of these organisms.

Many of the more modern techniques (so-called next generation sequencing techniques) are only able to sequence short nucleic acid molecules accurately. Next generation sequencing techniques can be used to sequence longer nucleic acid sequences, but this is often difficult and expensive. Next generation sequencing techniques can be used to generate short sequence reads, corresponding to sequences of portions of the nucleic acid molecule, and the full sequence can be assembled from the short sequence reads. Where the nucleic acid molecule comprises repeat regions, it may be unclear to the user whether two sequence reads having similar sequences correspond to sequences of two repeats within a longer sequence, or two replicates of the same sequence. Similarly, the user may want to sequence two similar nucleic acid molecules simultaneously, and it may be difficult to determine whether two sequence reads having similar sequences correspond to sequences of the same original nucleic acid molecule or of two different original nucleic acid molecules.

Assembling sequences from short sequence reads can be aided using sequencing aided by mutagenesis (SAM) techniques. In general SAM involves introducing mutations into target template nucleic acid sequences. The mutation patterns that are introduced may assist the user of the method in assembling the sequences of nucleic acid molecules from short sequence reads.

For example, where the template nucleic acid molecules contain repeat regions, the repeats may be distinguished from one another by different mutation patterns, thereby enabling the repeat regions to be resolved and assembled correctly.

In general, SAM techniques involve mutating copies of a target template nucleic acid molecule to produce a mutated target template nucleic acid molecule and/or one or more sequences comprising mutations, sequencing the one or more sequences comprising mutations to create SAM data including mutated sequence reads, and then assembling sequences from the mutated sequence reads based on their mutation patterns. Since the different mutated copies will comprise mutations at different positions, the assembled sequence may be representative of the original template nucleic acid molecule.

However, there remains a need for more reliable and/or more computationally efficient methods for processing SAM data.

### SUMMARY OF THE INVENTION

The present inventors have developed new improved methods for processing SAM data including mutated sequence reads. Thus, in an aspect of the invention, there is provided a computer-implemented method for determining a measure correlated to the probability that two mutated sequence reads derive from the same sequence comprising mutations. The method comprises receiving a plurality of mutated sequence reads. Each mutated sequence read corresponds to a subsequence of a sequence comprising mutations. The sequence comprising mutations comprises mutations compared to a sequence not comprising mutations. The method further comprises applying a common minimizer function to each mutated sequence read, thereby determining one or more respective minimizers for each mutated sequence read. The method further comprises determining positions of the one or more respective minimizers in each mutated sequence read. The method further comprises determining positions of one or more mutations in each mutated sequence read. The method further comprises, for at least two mutated sequence reads with a common minimizer, counting the number of mutations with matching position and/or with mismatching position when the respective minimizers are aligned.

In another aspect of the present invention, there is provided a method for generating at least a portion of a sequence of a target template nucleic acid molecule.

In another aspect of the present invention, there is provided a method for determining at least a portion of a sequence of at least one target template nucleic acid molecule.

Further aspects of the invention are provided in the dependent claims and in the detailed description.

### BRIEF DESCRIPTION OF THE FIGURES

FIG. 1 depicts an embodiment of a method for determining at least a portion of at least one target template nucleic acid molecule according the present invention.

FIG. 2 depicts an embodiment of a method for determining a measure correlated to the probability that two mutated sequence reads derive from the same sequence comprising mutations according to the present invention.

FIG. 3 depicts an example of a step of determining the positions of one or more mutations in a mutated sequence read.

FIG. 4A shows a comparative example of short read assembly of the 2.3 Mbp Arcobacter butzlerii genome not using the method of the present invention.

FIG. 4B shows an example of assembling a 2.3 Mbp Arcobacter butzlerii genome using the method of the present invention.

FIG. 5 shows experimental data on the effect of depth of short read coverage per long template on the results of the method of the present invention.

### DETAILED DESCRIPTION OF THE INVENTION

### General Definitions

Unless defined otherwise, technical and scientific terms used herein have the same meaning as commonly understood by a person skilled in the art to which this invention belongs.

In general, the term “comprising” is intended to mean including, but not limited to. For example, the phrase “a method comprising [certain steps]” should be interpreted to mean that the method includes the recited steps, but that additional steps may be performed.

In some embodiments of the invention, the word “comprising” is replaced with the phrase “consisting of”. The term “consisting of” is intended to be limiting. For example, the phrase “a method consisting of [certain steps]” should be understood to mean that the method includes the recited steps, and that no additional steps are performed.

In some aspects, the invention provides a method for determining or generating at least a portion of a sequence of at least one target template nucleic acid molecule. The method may be used to determine or generate a complete sequence of the at least one target template nucleic acid molecule. Alternatively, the method may be used to determine or generate a partial sequence, i.e. a sequence of a portion of the at least one target template nucleic acid molecule. For example, if it is not possible or not straightforward to determine a complete sequence, the user may decide that the sequence of a portion of the at least one target template nucleic acid molecule is useful or even sufficient for his purpose.

For the purposes of the present invention, a “nucleic acid molecule” (or “non-mutated nucleic acid molecule”) refers to a polymeric form of nucleotides of any length. The nucleotides may be deoxyribonucleotides, ribonucleotides or analogs thereof. Preferably, the at least one nucleic acid molecule is made up of deoxyribonucleotides or ribonucleotides. Even more preferably, the at least one nucleic acid molecule is made up of deoxyribonucleotides, i.e. the at least one nucleic acid molecule is a DNA molecule.

A “target template nucleic acid molecule” can be any nucleic acid molecule which the user would like to sequence. The at least one “target template nucleic acid molecule” can be single stranded, or can be part of a double stranded complex. If the at least one target template nucleic acid molecule is made up of deoxyribonucleotides, it may form part of a double stranded DNA complex. In which case, one strand (for example the coding strand) will be considered to be the at least one target template nucleic acid molecule, and the other strand is a nucleic acid molecule that is complementary to the at least one target template nucleic acid molecule. The at least one target template nucleic acid molecule may be a DNA molecule corresponding to a gene, may comprise introns, may be an intergenic region, may be an intragenic region, may be a genomic region spanning multiple genes, or may, indeed, be an entire genome of an organism.

For the purposes of the present invention, a “mutated nucleic acid molecule” or a “mutated target template nucleic acid molecule” refers to a “nucleic acid molecule” or a “target template nucleic acid molecule” into which mutations have been introduced. The mutations may be substitution mutations, optionally transition mutations. For the purposes of the present invention, the term “substitution mutation” should be interpreted to mean that a nucleotide is replaced with a different nucleotide. For example, the conversion of the sequence ATCC to the sequence AGCC introduces a single substitution mutation. For the purposes of the present invention, the term “transition mutation” should be interpreted to mean that the nucleotide A is replaced with the nucleotide G and vice versa (i.e. mutations A⇔G), or that the nucleotide C is replaced with the nucleotide T and vice versa (i.e. mutations C⇔T).

The phrase “introducing mutations into the at least one target template nucleic acid molecule” refers to exposing the at least one target template nucleic acid molecule in the second of the pair of samples to conditions in which the at least one target template nucleic acid molecule is mutated. This may be achieved using any suitable method. For example, mutations may be introduced by chemical mutagenesis and/or enzymatic mutagenesis.

For the purposes of the present invention, a “sequence comprising mutations” corresponds to at least a portion of the sequence of nucleotides in a “mutated nucleic acid molecule” or a “mutated target template nucleic acid molecule”. The “sequence comprising mutations” may also be referred to as a “mutated sequence”. A “sequence comprising mutations” is herein denoted as μⁱ, and a plurality of (i.e. multiple) “sequences comprising mutations” is denoted as M, where μ¹ . . . μⁿ∈M. A “sequence not comprising mutations” corresponds to at least a portion of the sequence of nucleotides in a “nucleic acid molecule” or a “target template nucleic acid molecule”. The “sequence not comprising mutations” may also be referred to as a “non-mutated sequence”. A “sequence not comprising mutations” is herein denoted as Sⁱ, and a plurality of (i.e. multiple) “sequences not comprising mutations” is denoted as S, where S¹ . . . Sⁿ∈S. The “sequence comprising mutations” and the “sequence not comprising mutations” thus may correspond to at least a portion of a nucleic acid molecule sequence of the nucleotides (nt) adenine (A), thymine (T), guanine (G) and cytosine (C). Such a chromosome sequence may range in size from 10³ to 10⁹ nucleotides (nt) in length and beyond.

For the purposes of the present invention, a “mutated sequence read” corresponds to a subsequence of the “sequence comprising mutations”, i.e. the “mutated sequence read” may be substantially identical to at least a subsequence of the “sequence comprising mutations”, but comprises mutations compared to the sequence comprising mutations and may comprise additional small differences due to read errors. A “mutated sequence read” is denoted as ρⁱ, and a plurality of (i.e. multiple) “mutated sequence reads” is denoted by P, where ρ¹ . . . ρⁿ∈P. A “non-mutated sequence read” corresponds to a subsequence of the “sequence not comprising mutations”, i.e. the “non-mutated sequence read” may be substantially identical to a subsequence of the “sequence not comprising mutations”, except for read errors during sequencing. A “non-mutated sequence read” is denoted as rⁱ, and a plurality of (i.e. multiple) “non-mutated sequence reads” is denoted by R, where rⁱ . . . rⁿ∈R. A “mutated sequence read” may be obtained by sequencing a region of a “mutated target template nucleic acid molecule”, and a “non-mutated sequence read” may be obtained by sequencing a region of a “target template nucleic acid molecule”. A sequence read may have a length that is less than a sequence, e.g. a length of about 150 nt.

Sequence Analysis Method 10

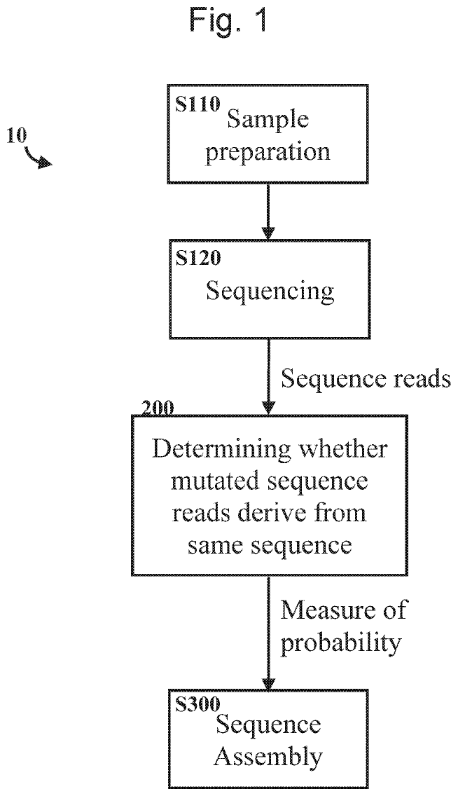

FIG. 1 shows a method 10 of determining at least a portion of at least one target template nucleic acid molecule in accordance with the invention.

The method 10 of determining at least a portion of at least one target template nucleic acid molecule may comprise a step S110 of sample preparation. The step S110 of sample preparation may include providing a pair of target template nucleic acid molecules, and introducing mutations into one of the pair of target template nucleic acid molecules so as to create a mutated target template nucleic acid molecule. The step S110 of sample preparation may include any known techniques for providing a target template nucleic acid molecule and a mutated target template nucleic acid molecule.

The method 10 of determining at least a portion of at least one target template nucleic acid molecule may further comprise a step S120 of sequencing. The step S120 of sequencing comprises sequencing regions of at least one target template nucleic acid molecule comprising mutations, thereby providing a plurality of mutated sequence reads P. In addition, the step S120 of sequencing may comprise sequencing regions of the at least one (non-mutated) target template nucleic acid molecule (a target template nucleic acid molecule that corresponds to the mutated target template nucleic acid molecule), thereby providing a plurality of non-mutated sequence reads R. The step S120 may comprise any known techniques for generating a plurality of mutated sequence reads P.

The method 10 of determining at least a portion of at least one target template nucleic acid molecule comprises a step 200 or method 200 of determining whether two mutated sequence reads ρⁱ, ρⁱ derive (or originate) from the same sequence comprising mutations μⁱ. Determining whether the two mutated sequence reads ρⁱ, ρⁱ derive (or originate) from the same sequence comprising mutations μⁱ comprises determining whether two mutated sequence reads ρⁱ, ρⁱ derive (or originate) from the same or a similar or overlapping portion of the sequence comprising mutations μⁱ, i.e. whether the two mutated sequence reads ρⁱ, ρⁱ both comprise a subsequence that corresponds to the same portion of the sequence comprising mutations μⁱ. The method 200 is a computer-implemented method, and may be carried out by a processor of a computer. The method 200 creates a measure correlated to the probability that two mutated sequence reads ρⁱ, ρⁱ derive from the same sequence comprising mutations μⁱ.

The method 10 of determining at least a portion of at least one target template nucleic acid molecule may further comprise a step S300 of sequence assembly. The step S300 of sequence assembly comprises assembling or reconstructing at least a portion of a sequence μⁱ, Sⁱ. The sequence comprising mutations μⁱ may be obtained by assembling the plurality of mutated sequence reads P based on the measure correlated to the probability that respective two mutated sequence reads ρⁱ, ρⁱ derive from the same sequence comprising mutations μⁱ.

This may be achieved, for example, by grouping the plurality of mutated sequence reads P into groups corresponding to the sequences comprising mutations μⁱ, and then assembling each group separately to reconstruct part or all of the individual sequences comprising mutations μⁱ. The sequence not comprising mutations Sⁱ may be obtained by error correction of the sequence comprising mutations μⁱ, e.g. by inferring the most likely sequence not comprising mutations Sⁱ from the sequence comprising mutations μⁱ using the plurality of non-mutated sequence reads R. The step S300 of sequence assembly may include any known methods for assembling a sequence comprising mutations μⁱ from a plurality of mutated sequence reads P based on a measure correlated to the probability that respective two mutated sequence reads ρⁱ, ρⁱ derive from the same sequence comprising mutations μⁱ.

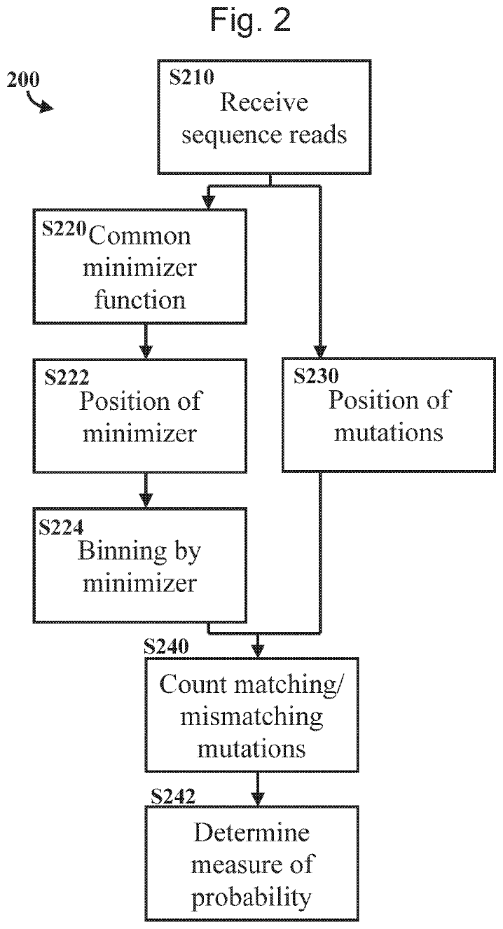

FIG. 2 shows a method 200 of determining whether two mutated sequence reads ρⁱ, ρⁱ derive from the same sequence comprising mutations μⁱ, in accordance with the present invention.

The method 200 comprises a step S210 of receiving a plurality of mutated sequence reads ρ¹ . . . ρⁿ∈P. Each mutated sequence read ρⁱ corresponds to a subsequence of a sequence comprising mutations μⁱ. The sequence comprising mutations μⁱ comprises mutations, for example substitution mutations, optionally transition mutations, compared to a sequence not comprising mutations Sⁱ. The sequence comprising mutations μⁱ may be at least a portion of the sequence of a mutated target template nucleic acid molecule, and the sequence not comprising mutations may be at least a portion of a (non-mutated) target template nucleic acid molecule, wherein the mutated target template nucleic acid molecule is obtained by introducing mutations, for example substitution mutations, optionally transition mutations, into the target template nucleic acid molecule. Each subsequence of a sequence comprising mutations μⁱ may be at least a portion of the sequence of a fragment of the mutated target template nucleic acid molecule. Each subsequence of a sequence not comprising mutations Sⁱ may be at least a portion of the sequence of a fragment of the target template nucleic acid molecule. The step S210 of receiving the plurality of mutated sequence reads P may comprise receiving the plurality of mutated sequence reads P directly from a sequencing machine used for sequencing the mutated target template nucleic acid molecule, or receiving the plurality of mutated sequence reads P from a data storage that stores the plurality of mutated sequence reads P.

The method 200 further comprises a step S220 of applying a common minimizer function to each mutated sequence read ρⁱ. Applying the common minimizer function determines one or more respective minimizers for each mutated sequence read ρⁱ. The method 200 further comprises a step S222 of determining positions of the one or more respective minimizers in each mutated sequence read ρⁱ.

In a preferred embodiment, the method 200 comprises a step S224 of binning the mutated sequence reads P by their respective minimizers. A mutated sequence read ρⁱ for which more than one minimizer has been determined may be provided in multiple respective minimizer bins.

The method 200 further comprises a step S230 of determining positions of one or more mutations in each mutated sequence read ρⁱ. The step S230 of determining positions of one or more mutations in each mutated sequence read ρⁱ may be carried out before, after or concurrently with the steps S220, S222 and S224 related to the common minimizer function.

The method 200 further comprises, for at least two mutated sequence reads ρⁱ, ρⁱ with a common minimizer, counting the number of mutations with matching position and/or with mismatching position when the respective minimizers are aligned, i.e. when the positions of the nucleotides of one mutated sequence read ρⁱ are shifted relative to the positions of the nucleotides of the other mutated sequence read ρⁱ such that the position of the minimizer of the one mutated sequence read ρⁱ is identical to the position of the minimizer of the other mutated sequence read pi. The number of mutations with matching position and/or with mismatching position may be the measure correlated to the probability that two mutated sequence reads derive from the same sequence comprising mutations. Alternatively, the method 200 may comprise a further step S242 of determining the measure correlated to the probability that two mutated sequence reads derive from the same sequence comprising mutations based on the number of mutations with matching position and/or with mismatching position.

Step S210 of Receiving a Plurality of Mutated Sequence Reads

Step S210 comprises receiving a plurality of mutated sequence reads ρⁱ . . . ρⁿ ∈P. Step S210 may further comprise receiving a plurality of non-mutated sequence reads r¹ . . . r^{m}∈R. Each mutated sequence read ρⁱ may correspond to a subsequence of a sequence comprising mutations μⁱ. Each non-mutated sequence read rⁱ may correspond to a subsequence of a sequence not comprising mutations Sⁱ.

The sequence comprising mutations may be obtained by introducing mutations into the sequence not comprising mutations Sⁱ. Each mutated sequence read ρⁱ may thus comprise mutations, i.e. correspond to a region of a mutated target template nucleic acid molecule that includes mutations, i.e. a subsequence of a sequence comprising mutations. In an embodiment, each mutated sequence read ρⁱ comprises substitution mutations, i.e. corresponds to the region of a mutated target template nucleic acid molecule that includes substitution mutations. In a preferred embodiment, the substitution mutations are transition mutations, so each mutated sequence read ρⁱ comprises transition mutations, i.e. corresponds to the region of a mutated target template nucleic acid molecule that includes transition mutations.

Each nucleotide of each sequence read ρⁱ, rⁱ is preferably encoded in binary format using two bits. It is advantageous, in particular when the plurality of mutated sequence reads P comprise transition mutations (A⇔G and C⇔T), that one of the two bits (e.g. the first bit) defines whether the nucleotide is a purine (A or G) or a pyrimidine (T or C). For example, the nucleotides may be encoded in binary form using the following format: A: 00, G: 01, C: 10 and T:11. This encoding will be used throughout this specification. However, it will be apparent that the invention is not limited to this encoding, and that the present invention could readily be carried out using any other encoding of the nucleotides.

Each sequence read ρⁱ, rⁱ may be encoded to account for homopolymer errors in the sequence read ρⁱ, rⁱ. Homopolymer errors arise when runs of the same nucleotide are misread at the wrong length, e.g. the sequence TAAAAGC might be misread as TAAGC because the sequencing machine has difficulty distinguishing the number of A's in a run of multiple A. To account for such homopolymer errors, runs of multiple identical nucleotides may be encoded as a single instance of the nucleotide. Alternatively, homopolymer errors may be accounted for during subsequent processing (i.e. not the initial encoding) of sequence reads ρⁱ, rⁱ, e.g. by encoding any k-mers used in the method 200, and/or any seed patterns used in step S230, such that runs of multiple identical nucleotides are encoded as a single instance of the nucleotide.

Steps S220 and S222: Common Minimizer Function

A minimizer is a k-mer from a set of k-mers that satisfies a common minimizer function min(•) on the set of k-mers.

For the purposes of the present application, a k-mer is a nucleotide subsequence of length k. The k-mer starting at position i in a sequence S=[S₁, S₂, . . . , Sₙ₋₁, Sₙ] of length n is denoted as k(Sᵢ), where k(Sᵢ)=[Sᵢ, Sᵢ₊₁, . . . , Sᵢ₊ₖ₋₁]. The set of k-mers in the sequence S with starting positions between i and j is denoted as k(Sᵢ . . . Sⱼ). The minimizer from all k-mers with starting positions in the range i to j of the sequence S will be denoted as min(k(Sᵢ . . . Sⱼ)).

The common minimizer function min(•) is used to determine one or more minimizers (i.e. one or more representative k-mers) from a set of k-mers, preferably all or substantially all k-mers, comprised by a sequence read ρⁱ, rⁱ, i.e. k-mers, preferably all or substantially all k-mers, that exist in the sequence read ρⁱ, rⁱ. For the purposes of the present invention, the set of k-mers that exist in the sequence read ρⁱ, rⁱ may include the k-mers of the reverse complement of the sequence read ρⁱ, rⁱ. Preferably, each minimizer is a k-mer of length equal to or greater than 5 (i.e. a 5-mer or longer), preferably equal to or greater than 10 (i.e. a 10-mer or longer), further preferably equal to or greater than 15 (i.e. a 15-mer or longer). Each minimizer may be a k-mer of length less than 50, optionally less than 30, further optionally less than 25. When the common minimizer function min(•) is used to determine longer minimizers, it is more likely that the minimizer that is determined is representative of a specific portion of a sequence, i.e. the minimizer is less likely to occur in multiple separate and unrelated portions of the sequence. Setting an upper limit on the size of the minimizers reduces the risk that the minimizers contain sequencing errors.

The step S220 of applying the common minimizer function min(•) may comprise identifying, the one or more k-mers in the respective mutated sequence read ρⁱ that is or are listed first in an ordered list of possible k-mers. The one or more minimizers determined for the respective mutated sequence read ρⁱ may be the identified one or more k-mers. The ordered list of possible k-mers may comprise all or some possible k-mers in a predetermined order. Step S220 may comprise generating the ordered list of possible k-mers, or may not comprise generating the ordered list of possible k-mers (e.g. in situations in which no direct comparison with a list is required to determine the minimizer, as in some examples below).

For example, the common minimizer function min(•) may determine as the minimizer the k-mer with the integer minimum of the two bit binary encodings of all k-mers in the mutated sequence read ρⁱ. In other words, the common minimizer function min(•) may identify the k-mer that is listed first in a list of k-mers that are ordered by the integer value of their two bit binary encodings. For example, based on the binary encoding A: 00, G: 01, C: 10 and T:11, the common minimizer function may identify the 5-mer in a mutated sequence read that is listed first in the example ordered list AAAAA, AAAAG, AAAAC, AAAAT, AAAGA, AAAGG, . . . , CTTTC, CTTTT, TTTTT. For example, the exemplary mutated sequence read:

ACGGAAAGCGCTACGAGCGACTGATATTGAGCTACGTTCAGAGCC

comprises 5-mers ACGGA, CGGAA, GGAAA, . . . , AGAGC, GAGCC. The 5-mer AAAGC is listed first in the example ordered list above, and the common minimizer function min(•) would identify AAAGC as the minimizer of this exemplary mutated sequence read. It will be appreciated that, for this common minimizer function min(•), it is not necessary to actually generate the ordered list of possible k-mers to determine the minimizer of a set of k-mers.

Determining the integer minimum of the two bit binary encodings of all k-mers in the mutated sequence read ρⁱ is just one example of a common minimizer function min(•) that may be applied to the mutated sequence read ρⁱ to determine the minimizer. Any other common minimizer function min(•) may be used. For example, it is advantageous for the common minimizer function min(•) to randomize the ordering of the integer minimum function. One way to achieve such randomization is to first apply a bitwise logical XOR with an arbitrary bitvector to each k-mer comprised by the mutated sequence read ρⁱ, after which the integer minimum function can be used.

Alternatively, a predetermined set of possible k-mers may be used instead of the ordered list of possible k-mers, and applying the common minimizer function min(•) comprises identifying the one or more k-mers that exist in the predetermined set of possible k-mers. The one or more minimizers determined for the respective mutated sequence read ρⁱ may be the identified one or more k-mers. The predetermined set of possible k-mers may be ordered or unordered. The predetermined set of possible k-mers may be a set of k-mers that include only k-mers that are suitable or intended to be used as minimizers. The step S220 of applying the common minimizer function min(•) may comprise creating the predetermined set of possible k-mers.

In a preferred embodiment, in the ordered list of possible k-mers, the k-mers are ordered based on the probability that the k-mers exist in the sequence comprising mutations and do not exist in the sequence not comprising mutations Sⁱ, i.e. k-mers that are relatively likely to exist in the sequence comprising mutations and not in the sequence not comprising mutations may be listed earlier in the ordered list, and k-mers that are relatively unlikely to exist in the sequence comprising mutations and not in the sequence not comprising mutations may be listed later in the ordered list. In an alternative preferred embodiment, the predetermined set of possible k-mers comprises k-mers that are relatively likely to exist in the sequence comprising mutations and not in the sequence not comprising mutations, and optionally does not comprise k-mers that are relatively unlikely to exist in the sequence comprising mutations and not in the sequence not comprising mutations. The step S220 may comprise determining which k-mers comprised by the plurality of mutated sequence reads P are relatively likely to exist in the sequence comprising mutations and not in the sequence not comprising mutations, for example by comparing the number of occurrences (or observations) of a k-mer in the plurality of mutated sequence reads P to the number of occurrences of the k-mer in the plurality of non-mutated sequence reads R. This may comprise counting the number of occurrences of the k-mer in the plurality of mutated sequence reads P, and counting the number of occurrences of the k-mer in the plurality of non-mutated sequence reads R.

In both preferred embodiments, the common minimizer function min(•) is chosen so as to preferentially, determine as the one or more minimizers, k-mers that are more likely to occur in a mutated sequence read ρⁱ than in a non-mutated sequence read rⁱ. This increases the likelihood that each minimizer comprises a mutation.

In a more preferred embodiment, the ordered list of possible k-mers contains only, i.e. consists of, k-mers that exist more often in the plurality of mutated sequence reads P than in the plurality of non-mutated sequence reads R (or more often in the sequence comprising mutations than in the sequence not comprising mutations), i.e. k-mers for which the number of occurrences in the plurality of mutated sequence reads P is greater than the number of occurrences in the plurality of non-mutated sequence reads R. In an alternative more preferred embodiment, the predetermined set of possible k-mers contains only, i.e. consists of, k-mers that exist more often in the plurality of mutated sequence reads P than in the plurality of non-mutated sequence reads R (or more often in the sequence comprising mutations than in the sequence not comprising mutations), i.e. k-mers for which the number of occurrences in the plurality of mutated sequence reads P is greater than the number of occurrences in the plurality of non-mutated sequence reads R. Preferably, the ordered list of possible k-mers or the predetermined set of possible k-mers contains only, i.e. consists of, k-mers that exist n or more times in the plurality of mutated sequence reads and exist fewer than n times in the plurality of non-mutated sequence reads, i.e. k-mers for which the number of occurrences in the plurality of mutated sequence reads P is equal to or greater than n, and the number of occurrences in the plurality of non-mutated sequence reads R is fewer than n. n may be an integer greater or equal to 1. n may be an integer greater or equal to 2. Preferably, n is 2. Further preferably, the ordered list of possible k-mers or the predetermined set of possible k-mers contains only, i.e. consists of, k-mers that do not exist in the plurality of non-mutated sequence reads, i.e. k-mers for which the number of occurrences in the plurality of non-mutated sequence reads R is 0.

For example, the ordered list of possible k-mers or the predetermined set of possible k-mers may contain only k-mers that exist at least twice in the set of k-mers of the plurality of mutated sequence reads P, but exist never (or rarely) in the set of k-mers of the plurality of non-mutated sequence reads R. This ensures that, with high probability, the ordered list of possible k-mers or the predetermined set of possible k-mers includes minimizers that contain a mutation that is present in two or more of the plurality of mutated sequence reads P. Optionally, k-mers that exist more often in the plurality of mutated sequence reads than in the plurality of non-mutated sequence reads are relatively likely to exist in the sequence comprising mutations. Optionally, wherein k-mers that exist n or more times in the plurality of sequence reads and exist less than n times in the plurality of non-mutated sequence reads are relatively likely to exist in the sequence comprising mutations.

The predetermined set of possible k-mers may be created by building a set of mutation minimizers U_{M}, where U_{M} comprises k-mers, preferably all or substantially all k-mers, for which the number of occurrences or observations in the plurality of mutated sequence reads P is greater than or equal to n (preferably where n≥2, more preferably where n is 2) and the number of occurrences or observations in the plurality of non-mutated sequence reads P is less than n (preferably where n is 0 or 1, more preferably where n is 0). The set of mutation minimizers U_{M} may be created by counting how often each k-mer exists in the plurality of non-mutated sequence reads R and the plurality of mutated sequence reads P. The set of mutation minimizers U_{M} may be calculated efficiently from the plurality of non-mutated sequence reads R and the plurality of mutated sequence reads P using probabilistic data structures, such as a counting Bloom filter, or the related cuckoo and quotient filter methods. The ordered list of possible k-mers may be created from the entire set of mutation minimizers U_{M}.

The set of mutation minimizers U_{M} may be used as the predetermined set of possible k-mers. Alternatively, the set of mutation minimizers U_{M} may be processed further to create the predetermined set of possible k-mers. In a preferred embodiment, a subset W_{M} of the set of mutation minimizers U_{M} is used as the predetermined set of possible k-mers. The subset W_{M} may be constructed by splitting each mutated read ρⁱ∈P into two or more non-overlapping sections (optionally of substantially equal size), e.g. non-overlapping sets of k-mer starting positions of size L_{w}, e.g. {1 . . . L_{w}}, {L_{w}+1 . . . 2L_{w}}, etc. A typical value for L_{w} may be 50 when mutated sequence reads of length 150 are in use, thereby splitting the possible k-mer start positions into 3 groups. Then for each set of starting positions, the subset W_{M} can be denoted as follows:

$${W}_{M} = {⋃}_{ρ ∈ P} {⋃}_{j = 1 … ⌈ ℒ / {ℒ}_{w} ⌉} min ( k ( {ρ}_{( j - 1 ) {ℒ}_{w} + 1} … {ρ}_{j {ℒ}_{w}} ) ⋂ {U}_{M} )$$

In effect, each of the plurality of mutated sequence reads P may be split into two or more sections (e.g. 3 sections) and a minimizer may be found to represent each section. The minimizer is determined by first finding the candidate minimizers via the intersection of k-mers in that section of the respective mutated sequence read with the set of mutation minimizers U_{M}, and then applying a common minimizer function to that set to identify one minimizer for each section.

As such, in a preferred embodiment, the step S220 of applying a common minimizer function min(•) to each mutated sequence read comprises:
creating a set of mutation minimizers U_{M} that consists of k-mers, preferably all or substantially all k-mers, in the plurality of mutated sequence reads P that exist n or more times in the plurality of mutated sequence reads P and exist less than n times in the plurality of non-mutated sequence reads R, where n is an integer greater or equal to 2;
optionally creating a subset W_{M} of the set of mutation minimizers U_{M} by splitting each of the plurality of mutated sequence reads P into two or more sections, identifying k-mers, preferably all or substantially all k-mers, in each section of each of the plurality of mutated sequence reads P that exist in the set of mutation minimizers U_{M}, and adding to the subset W_{M} one of the identified k-mers for each section of each of the plurality of mutated sequence reads P, optionally wherein the one of the identified k-mers for each section of each of the plurality of mutated sequence reads P is selected by applying a common minimizer function min(•) (e.g. finding the integer minimum or any other known minimizer function) to the identified k-mers for each section of each of the plurality of mutated sequence reads P; and
using the set of mutation minimizers U_{M}, or a subset of the set of mutation minimizers U_{M} (such as the subset W_{M}) as the predetermined set of possible k-mers, and, for each of the plurality of mutated sequence reads P, identifying k-mers, preferably all or substantially all k-mers, in the respective mutated sequence read μ¹ that exist in the predetermined set of possible k-mers, wherein the one or more minimizers determined for a respective mutated sequence read are the identified k-mers.

The method 200 further comprises the step S222 of determining positions j of the one or more respective minimizers in each mutated sequence read ρⁱ. The positions j of each of the minimizers in each respective mutated sequence read ρⁱ may be stored as an integer bit value in association with (e.g. in the same location or minimizer bin as) the respective minimizer.

Step S224: Minimizer Binning

In a preferred embodiment, the method 200 comprises the step S224 of binning the mutated sequence reads P in one or more minimizer bins. Binning the mutated sequence reads P in one or more minimizer bins comprises binning an index i characteristic of the mutated sequence read p′ in one or more minimizer bins. Each minimizer bin may contain mutated sequence reads P having a common minimizer, and not contain mutated sequence reads P not having the common minimizer. The step S240 of counting the number of mutations with matching position and/or with mismatching position may be performed only on mutated sequence reads P in the same minimizer bin. This improves the computational efficiency of performing the step S240.

In other words, the one or more minimizers may be used as hash keys, to collect sequence reads containing the minimizer in a common hash bucket (herein referred to as a minimizer bin), for example in preparation for some additional processing (e.g. step S240) on those sequence reads.

Each minimizer that is determined by applying the common minimizer function min(•) to the mutated sequence reads P may be used for the purpose of binning the mutated sequence reads P in the one or more minimizer bins. In an embodiment, each minimizer in the ordered list of possible k-mers, or each minimizer in the predetermined set of possible k-mers (e.g. each minimizer in the set of mutation minimizers U_{M}, or a subset thereof, such as the subset W_{M}), may be used for the purpose of binning the mutated sequence reads P in the one or more minimizer bins.

The step S224 of binning the mutated sequence reads P in one or more minimizer bins may comprise creating the one or more minimizer bins. This may comprise creating one minimizer bin for each minimizer determined by the common minimizer function min(•), or one minimizer bin for each minimizer (or k-mer) in the predetermined set of possible k-mers U_{M}, or one minimizer bin for each k-mer in the subset W_{M}. Each minimizer bin may be implemented as a contiguous block of RAM. Preferably, collections of minimizer bins are implemented as a file on a computer storage medium (such as a computer disk, e.g. a spinning magnetic disk or a solid state disk), allowing each bin to store large amounts of data (as appropriate in sequence analysis).

The step S224 of binning the mutated sequence reads P in one or more minimizer bins may comprise storing the mutated sequence read ρⁱ, or an index i characteristic of the mutated sequence read ρⁱ, in the respective minimizer bin. The step S222 of determining positions j of the one or more respective minimizers in each mutated sequence read ρⁱ may comprise storing the position j of the respective minimizer in the respective minimizer bin. In addition, the position α=morphomuts(ρⁱ,V_{R}) of the one or more mutations in each mutated sequence read ρⁱ, as determined by the step S230 of determining positions α of one or more mutations in each mutated sequence read, may be stored in the respective minimizer bin. Optionally, arbitrary additional values, such as the sequence of the mutated sequence read ρⁱ, quality information about the accuracy of the sequence, or other information, could be stored in the minimizer bin if they are useful for downstream processing. These values associated with each mutated sequence read ρⁱ may be stored as a tuple in each minimizer bin. For notational purposes, the tuple elements of the y-th element of the z-th minimizer bin b_{z,y} are denoted as b_{z,y}.i, b_{z,y}.j, and b_{z,y}.α. Each mutated sequence read ρⁱ may be added to multiple minimizer bins.

Step S230: Positions of Mutations

The method 200 comprises the step S230 of determining the positions α of the one or more mutations in each mutated sequence read ρⁱ. The step S230 of determining the positions α of the one or more mutations in each mutated sequence read ρⁱ may be performed using alignment-free methods.

The step S230 of determining the positions α of the one or more mutations in each mutated sequence read ρⁱ may comprise obtaining a set of non-mutated seed-masked k-mers V_{R}, i.e. a set of k-mers of the non-mutated sequence reads R to which one or more seed patterns ψ have been applied. Obtaining the set of non-mutated seed-masked k-mers V_{R} may comprise creating or generating the set of non-mutated seed-masked k-mers V_{R}. The set of non-mutated seed-masked k-mers V_{R} may be obtained or created by applying each one of one or more seed patterns to each k-mer in the sequence not comprising mutations, e.g. each k-mer in the non-mutated sequence reads. Applying a seed pattern to a k-mer may comprise determining the bit-wise logical AND of the seed pattern and the (two-bit encoded) k-mer. Applying a seed pattern to a k-mer creates a seed-masked k-mer. The set of seed-masked non-mutated k-mers V_{R} may be denoted as

V_{R}{∀_{r(i)∈R} [symbol] :ψ(k(rⱼⁱ))},

i.e. the set of seed-masked non-mutated k-mers V_{R} is created by applying each of one or more seed pattern ψ of a seed family Ψ to each k-mer k(rⱼⁱ), for all (or substantially all) positions j of the k-mer (i.e. from 1 to [symbol] -k) in each non-mutated read rⁱ, for all (or substantially all) non-mutated reads rⁱ in the plurality of non-mutated sequence reads R.

A seed pattern may be used to modify the process of comparing k-mers to each other. A seed pattern is defined as a set of positions (i.e. the nucleotides) within two k-mer which are required to be identical in both k-mers in order to consider the seed-masked k-mers to be matching. The seed pattern may comprise masking positions and non-masking positions. Applying the seed patterns to a k-mer creates a seed-masked k-mer, where the positions of the seed-masked k-mer corresponding to the masking positions of the corresponding seed pattern are ignored in any further processing (such as comparisons), whereas the positions of the seed-masked k-mer corresponding to the non-masking positions of the corresponding seed pattern are not ignored in any further processing (such as comparisons). For example, the seed pattern {1,2,4,6,7} would require the first, second, fourth, sixth, and seventh positions (or nucleotides) in two k-mers k(Sᵢ) and k(Sⱼ) under comparison to be identical in order to consider them as matching (for k=7). The third and fifth positions in the two k-mers could be arbitrary nucleotides. This means that the third and fifth positions in the two seed-masked k-mers are masked by the seed pattern.

Optionally, the one or more seed patterns may be one or more transition seed patterns. This is advantageous in particular when the sequence comprising mutations M comprises transition mutations compared to the sequence not comprising mutations S, i.e. each of the plurality of mutated sequence reads P contains one or more transition mutations.

A transition seed pattern is a specialized type of seed pattern where positions fall into three classes instead of just two: each position can be (1) required to match exactly, or (2) both be purines or be pyrimidines, or (3) be any of the four nucleotides, in order to match. Transition seed patterns are particularly advantageous when the sequence comprising mutations comprises transition mutations. When implemented on a computer using the two bit nucleotide encoding introduced above, a position required to match exactly can be implemented as the bit mask 11, while a position where only transition mutations are allowed as 10, and a position where any nucleotide is allowed as 00. The seed pattern {1,2,4,6,7} may be written as the bitmask 11110011001111. The transition seed pattern {1,2,4,6,7} may be written as the bitmask 11111011101111. Two k-mers can be evaluated for a match by computing the bitwise logical AND of the bitmask and the two bit encoding of the k-mers individually, and then checking for identity of the two resulting seed-masked k-mers. For convenience, the function that applies a seed pattern to a k-mer k(Sᵢ) via bitwise logical AND will be denoted as the function ψ(k(Sᵢ)).

In an embodiment, the one or more seed patterns are chosen such that the probability of obtaining identical seed-masked k-mers by applying at least one of the one or more seed patterns to any k-mer in the plurality of mutated sequence reads P (or the sequence comprising mutations) and to a corresponding k-mer in the plurality of non-mutated sequence reads R (or the sequence not comprising mutations) is greater than 90%, preferably greater than 95%, further preferably greater than 98%, most preferably greater than 99%.

The one or more seed patterns may make up a seed family Ψ. A seed family Ψ is a collection of two or more seed patterns which when used together, are able to identify matches among k-mers at a particular percent nucleotide identity with high probability, for example a probability greater than 90%, preferably greater than 95%, further preferably greater than 98%, most preferably greater than 99%. A seed family Ψ is denoted as a set of n different functions to apply seed patterns ψ₁ . . . ψₙ∈Ψ. The weight of a seed pattern w(ψ) is defined as the number of positions the seed requires to be identical in order for two k-mers to be considered matching, where w(ψ)≤k.

The step S230 of determining the positions α of the one or more mutations in each mutated sequence read ρⁱ may comprise, for each mutated sequence read, applying each one of the one or more seed patterns ψᵢ to k-mers (optionally each k-mer) in the respective mutated sequence read ρⁱ to obtain a plurality of mutated seed-masked k-mers. The positions of the one or more mutations may be determined by identifying the one or more positions in the mutated sequence read ρⁱ that are masked by all of the seed patterns that correspond to the mutated seed-masked k-mers of the plurality of mutated seed-masked k-mers that exists in the set of non-mutated seed-masked k-mers V_{R}. This means that the positions that are not mutations in the mutated sequence read ρⁱ may be identified as the one or more positions in the mutated sequence read ρⁱ that that are not masked by any one of the seed patterns that correspond to the mutated seed-masked k-mers of the plurality of mutated seed-masked k-mers that exists in the set of non-mutated seed-masked k-mers V_{R}.

For example, the positions α of the one or more mutations of each mutated sequence read ρⁱ may be determined by:
creating a bitvector α of length 2L and initializing the bitvector α to 0s
creating a bitvector b of length 2k initializing the bitvector b to all is
for each ψ∈Ψ, and for each position j in the read between 1 and [symbol] -k, compute ψ(k(ρⱼⁱ)). If ψ(k(ρⱼⁱ))∈V_{R} then assign α←α|(ψ(b)>>2j), where the operator | denotes the bitwise logical OR operator, and the operator >> denotes the bit shift right operator. The set membership query in V_{R} can be implemented either exactly using something like a hash table, or approximately using a high efficiency probabilistic data structure like a Bloom filter, Quotient filter or similar approach.
Optionally, for ease of further processing, transforming the bit vector a from the length of the binary two bit mutated sequence read encoding to the length of the mutated sequence read itself by removing the odd positions, e.g. α←{α2, α4, α6, . . . α2L}.
Optionally, for ease of further processing, applying a logical NOT to flip the bits so that 1's represent positions for which a seed match was never found.

The result of the above procedure will be a bit vector a where each position containing a 1 corresponds to the position of a mutation with high probability. For notational purposes, the function that computes the bit vector a for a mutated sequence read ρⁱ is denoted as α=morphomuts(ρⁱ,V_{R}).

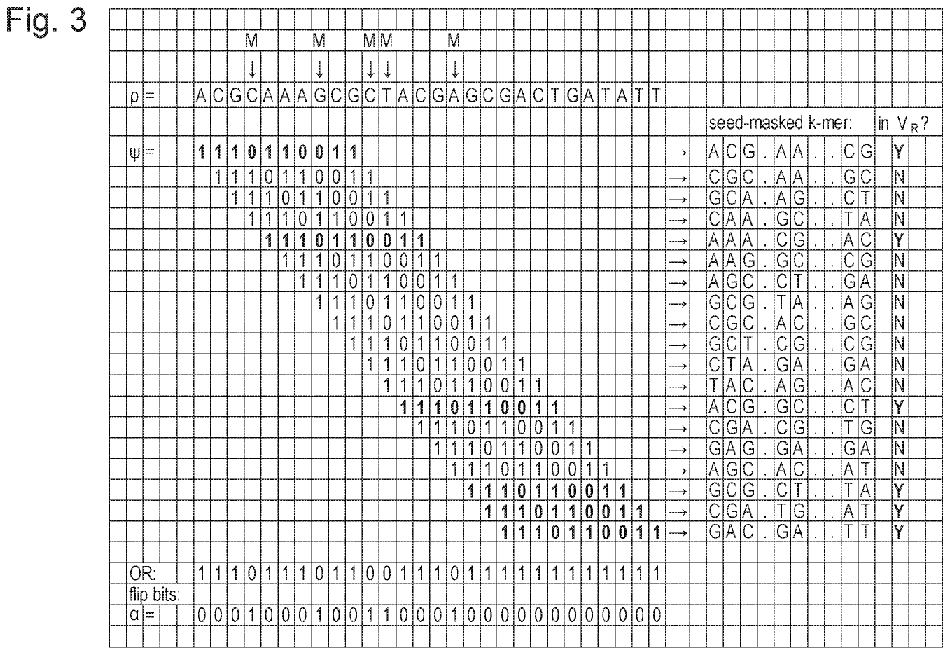

FIG. 3 shows an example that illustrates how the bit vector a may be obtained for an example mutated sequence read ρ=ACGCAAAGCGCTACGAGCGACTGATATT using a single seed pattern ψ=1110110011. The 4^{th}, 8^{th}, 11^{th}, 12^{th} and 16^{th} positions of the mutated sequence read ρ correspond to mutations in the mutated sequence read p, i.e. the nucleotides in these positions will be different in the sequence not comprising mutations. In practice, the mutated sequence read ρ may be encoded in two-bit binary format, and each position of the seed pattern ψ may cover two bits (i.e. each 1 in the seed pattern ψ would be implemented as two binary 1s, and each 0 in the seed pattern ψ would be implemented as a binary 00 or a binary 10). The set of seed-masked non-mutated k-mers V_{R} was previously generated in this example.

As shown in the example of FIG. 3, the seed pattern ψ is applied to each k-mer in the mutated sequence read ρ, thereby creating one seed-masked k-mer for each k-mer in the mutated sequence read ρ. It is then checked whether the seed-masked k-mer exists in the set of seed-masked non-mutated k-mers V_{R}. In the example shown, the 1^{st}, 5^{th}, 13^{th}, 17^{th}, 18^{th} and 19^{th} seed-masked k-mers all exist in the set of seed-masked non-mutated k-mers V_{R}. These seed-masked k-mers do not contain a mutation position that is not masked by the seed pattern.

The 1^{st}, 5^{th}, 13^{th}, 17^{th}, 18^{th} and 19^{th} seed-masked k-mers are then used to identify the positions that are masked by all of the seed patterns corresponding to these seed-masked k-mers. The 4^{th} position of the mutated sequence read ρ is masked by all of these seed pattern, noting that the 4^{th} position of the mutated sequence read ρ is ignored when processing the 13^{th}, 17^{th}, 18^{th} and 19^{th} seed-masked k-mers, i.e. the 4^{th} position of the mutated sequence read ρ is masked by the seed pattern of 13^{th}, 17^{th}, 18^{th} and 19^{th} seed-masked k-mers. None of these seed patterns do not mask the 4^{th} position of the mutated sequence read ρ. The 4^{th} position of the mutated sequence read ρ is thus identified as the position of a mutation. By contrast, while the 7^{th} position of the mutated sequence read ρ is masked by all of the seed patterns corresponding to the 1^{st}, 13^{th}, 17^{th}, 18^{th} and 19^{th} seed-masked k-mers, this 7^{th} position of the mutated sequence read ρ is not masked by the seed pattern corresponding to the 5^{th} seed-masked k-mer. As such, the 7^{th} position of the mutated sequence read ρ is not identified as a position of a mutation. Instead, the 7^{th} position of the mutated sequence read ρ is identified as a position that is not a mutation.

Effectively, all of the seed-patterns corresponding to the 1^{st}, 5^{th}, 13^{th}, 17^{th}, 18^{th} and 19^{th} seed-masked k-mers are combined using a logical OR. The bits of the resulting bitvector may be flipped (e.g. using a logical NOT operation) to obtain the positions of the mutations in the mutated sequence read ρ as the bitvector α.

### Alternative Implementation of Step 230 Using Reference Assembly

In the embodiment described above, the step 230 of determining the positions α of the one or more mutations in each mutated sequence read ρⁱ is performed using the plurality of mutated sequence reads P and the plurality of non-mutated sequence reads R, based on applying seed patterns to each mutated sequence read ρⁱ.

In large and complex genomes, such as the human genome, a significant fraction of the genome is comprised of repetitive sequences. For example, over half of the human genome is thought to be part of repetitive sequences. These repetitive sequences are classed into “families” of similar repetitive sequences. The most common family in the human genome is the Alu family of SINEs (Short Interspersed Nuclear Elements), which are around 300 nt long and present in around 1 million copies. Another common family is the L1 family of Long Interspersed Nuclear Elements (LINEs), with elements ranging in size from 1 to 6.5 kbp and with a copy number in the 10,000s.

The various copies of the repetitive sequences within the genome may be non-identical, for example containing single base differences. Due to the biology of mutation, those differences are often transition differences In some situations, these differences may appear similar to the differences due to the introduction of mutations between the plurality of mutated sequence reads P and the plurality of non-mutated sequence reads R. This is particularly true for certain polymerase-based approaches for mutagenesis used to introduce mutations into the at least one target template nucleic acid molecule as part of producing the plurality of mutated sequence reads P, as these often introduce transition mutations.

As a result, the plurality of non-mutated sequence reads R may contain a large number of k-mers that differ from one another only by some number of transition differences. Therefore, the plurality of mutated sequence reads P may include one or more k-mers that are identical to k-mers in the plurality of non-mutated sequence reads R, despite the presence of the mutations compared to the non-mutated sequence reads R. In some situations, it is possible that these naturally-occurring differences among different copies of the repetitive sequence in different non-mutated sequence reads r′ would partially “mask” the mutations introduced in the plurality of mutated sequence reads P. This is particularly pronounced with the Alu families of SINEs.

It would therefore be advantageous if, in situations such as these, an embodiment of the method were provided that could better discriminate intentionally introduced mutations from natural differences between copies of the repetitive sequences.

A first approach to improving the ability of the method to discriminate intentionally introduced mutations from natural differences between copies of the repetitive sequences is to use seed patterns with much higher weight, so that the mutated seed-masked k-mers become more likely to include one or more positions that contain a difference that distinguishes the copies of the repetitive sequence. In an embodiment adopting the first approach, the weight w(ψ) of each seed pattern ψ is in the range from 50 to 100, preferably in the range from 70 to 90. For the human genome, a weight of approximately 80 would be sufficient for the first approach.

The first approach may not be ideal in all cases, however. A seed pattern with weight 80 would be very long, likely longer than the typical length of the mutated sequence reads ρⁱ. In addition, the size of the seed family required to ensure high sensitivity might become very large, thus requiring significant additional computational resources to process all of the seed patterns. Finally, the probability of the seed pattern covering an indel error would grow, and additional algorithmic complexity would be required to accommodate the possibility of indel errors. Therefore, this first approach may not be preferred in some circumstances.

A second approach to improving the ability of the method to discriminate intentionally introduced mutations from natural differences between copies of the repetitive sequences is to use an approach based on aligning (or mapping) the plurality of mutated sequence reads P to a reference assembly (or reference genome). The reference assembly can either be independently generated, such as the hg38 human genome produced by the Genome Reference Consortium (GRC), or a de-novo assembly based on the plurality of non-mutated sequence reads R. In the second approach, the step of determining the positions of the one or more mutations in each mutated sequence read comprises for one or more mutated sequence reads, aligning the respective mutated sequence read to a reference assembly.

This approach may be particularly suitable when the mutated sequence reads ρⁱ are paired-ended sequence reads. The advantage of aligning paired-end mutated sequence reads to a reference assembly, particularly with respect to SINE repeats, is that the fragment size of a short read shotgun library is typically larger than the length of the repetitive sequences. A typical fragment size for paired-end sequencing is 400-600 bp, with about 150 bp sequenced from each end of the fragment. Thus, if one of the paired-end sequence reads in the pair of paired-end sequence reads lands in a repetitive sequence, the other of the paired-end sequence reads in the pair of paired-end sequence reads is likely to land in a unique sequence outside the repetitive sequence. Therefore, a standard paired-end aligning program (e.g. a Burrows-Wheeler aligner such as BWA-MEM) is able to confidently align the pair of paired-end sequence reads to the correct location in the reference assembly, including the correct copy of the repetitive sequence. It is then possible to record the positions of any differences between the aligned mutated sequence reads ρⁱ and the reference assembly, and store them in a bitvector α analogous to that derived using the approach based on applying seed patterns to each mutated sequence read ρⁱ. Thereby, determining the positions of the one or more mutations in the respective mutated sequence read is achieved by identifying positions in the respective mutated sequence read of differences between the respective mutated sequence read and the reference assembly.

However, aligning the plurality of mutated sequence reads P to a reference assembly may not be ideal in some situations, because any given target template nucleic acid molecule will typically have regions that are not represented in the reference assembly. Therefore, it is not possible to align mutated sequence reads ρⁱ to those regions that are not represented in the reference assembly and derive a bitvector α from differences between the aligned mutated sequence reads ρⁱ and the reference assembly. In addition, the regions that are not represented in the reference assembly are often of clinical interest as they represent Structural Variant insertions relative to the reference assembly. In addition to large insertion regions, any mutations that occur in small insertions relative to the reference assembly would also be missed by the approach based on aligning the plurality of mutated sequence reads P to a reference assembly.

Therefore, a third, hybrid approach to improving the ability of the method to discriminate intentionally introduced mutations from natural differences between copies of the repetitive sequences is to combine the approach based on aligning the plurality of mutated sequence reads P to a reference assembly with the approach based on applying seed patterns to each mutated sequence read ρⁱ. This third approach may be used as an alternative implementation of step 230 of the present method.

In the third approach, the position of the one or more mutations in each mutated sequence read are determined using both the approach based on aligning the plurality of mutated sequence reads P to a reference assembly and the approach based on applying seed patterns to each mutated sequence read ρⁱ. If a position in the respective mutated sequence read is aligned to the reference assembly, the position in the respective mutated sequence read is determined to be a position of a mutation in the respective mutated sequence read if the position in the respective mutated sequence read is a position at which the respective mutated sequence read differs from the reference assembly. If a position in the respective mutated sequence read is not aligned to the reference assembly, the position in the respective mutated sequence read is determined to be a position of a mutation in the respective mutated sequence read if the position in the respective mutated sequence read is a position that is masked by all of the seed patterns that correspond to the mutated seed-masked k-mers of the plurality of mutated seed-masked k-mers that exists in the set of non-mutated seed-masked k-mers.

To achieve this, a bitvector α of the type described above is derived independently via both the approach based on aligning and the approach based on applying seed patterns. The bitvector from the approach based on applying seed patterns to each mutated sequence read ρⁱ is denoted α_{mmd} and the bitvector from the approach based on aligning the plurality of mutated sequence reads P to a reference assembly is denoted αₘₐₚ. A further alignment mask bitvector denoted αₐₘₐₛₖ is also constructed that records the positions of each mutated sequence read that successfully aligned to the reference assembly. The alignment mask bitvector αₐₘₐₛₖ will have a 1 in each position that successfully aligned, and a 0 in positions that did not successfully align to the reference assembly.

A final hybrid bitvector α_{hybrid} is then constructed that combines the bitvector from the approach based on applying seed patterns to each mutated sequence read ρⁱ, α_{mmd} and the bitvector from the approach based on aligning the plurality of mutated sequence reads P to a reference assembly, αₘₐₚ as follows:

α_{hybrid}=αₘₐₚ|(α_{mend}&˜αₐₘₐₛₖ)

Where denotes the bitwise logical OR operator, & denotes bitwise logical AND, and ˜ denotes bitwise NOT.

The third approach thus uses the positions of mutations determined from aligning to the reference assembly in positions of the mutated sequence read where aligning was successful, and positions of mutations determined by applying seed patterns in all other positions. This has the advantage of enabling a high-quality reference assembly to be incorporated into the analysis, whilst also handling all types of insertions relative to the reference assembly. Aligning against an independent high quality reference assembly such as the human reference genome can be much more accurate than aligning against a de novo short read assembly. Using positions of mutations determined from aligning to the reference assembly can provide more accurate estimates of mutation positions, especially in repetitive sequence regions, while the alignment-free seed pattern based method can identify positions of mutations in regions that are not represented in the reference. The latter can happen without having to compute an assembly, which is a computationally-demanding task. The hybrid approach therefore provides improvements in the accuracy of identifying the positions of mutations and computational efficiency relative to the use of either approach individually.

It is also possible to “augment” a reference assembly with variants and locally assembled regions from a particular target template nucleic acid molecule to produce an assembly graph specific to that target template nucleic acid molecule. A bitvector from the approach based on aligning the plurality of mutated sequence reads P to a reference assembly (denoted αₘₐₚ) could be derived from aligning mutated sequence reads against that augmented assembly graph, and then combined with the approach based on applying seed patterns to each mutated sequence read ρⁱ for any regions of the target template nucleic acid molecule that remain difficult to align due to technical or other reasons.

Steps S240 and S240: Determining the Measure Correlated to the Probability that Two Mutated Sequence Reads Derive from the Same Sequence Comprising Mutations

The method 200 comprises the step S240 of, for at least two mutated sequence reads with a common minimizer, counting the number of mutations with matching position and/or with mismatching position when the respective minimizers are aligned.

This may be achieved by first determining the difference in the position j of the minimizer determined in step S222 for each of the two mutated sequence reads. For example, the difference in the position j of the minimizer for each of the two mutated sequence reads ρ^{a} and ρ^{c} stored in minimizer bin b_{z} as a=b_{z,y}.i and c=b_{z,x}.i may be determined as d=b_{z,y}.j−b_{z,x}.j.

Counting the number of mutations with matching positions may comprise determining the size of the set intersection of the positions of mutations determined in step S230 when the positions of mutations determined for one of the two mutated sequence reads are right shifted by d. For example, for the two mutated sequence reads ρ^{x} and ρ^{y} stored as b_{z,y} and b_{z,x}, the number of mutations with matching positions may be determined as:

λ_{x,y}=|Ω(b_{z,x}.α)∩(Ω(b_{z,y}.α)−d)|

where Ω(α) is defined as the set of position indexes in a which are nonzero (i.e. the set of positions of the mutations in the respective mutated sequence read ρⁱ), and where Ω(b_{z,y}.α)−d is understood to be the element-wise subtraction of d from Ω(b_{z,y}.α). The set intersection can be implemented efficiently on a computer using bit shift and popcount CPU instructions.

Counting the number of mutations with mismatching positions may comprise determining the size of the symmetric set difference of the positions of mutations determined in step S230 when the positions of mutations determined for one of the two mutated sequence reads are right shifted by d. For example, for the two mutated sequence reads ρ^{x} and ρ^{y} stored as b_{z,y} and b_{z,x}, the number of mutations with mismatching positions may be determined as:

δ_{x,y}=|Ω(b_{z,x}.α)\(Ω(b_{z,y}.α)−d))∪((Ω(b_{z,y}.α)−d)\Ω(b_{z,x}.α))|.

The step S242 of determining the measure correlated to the probability that the at least two mutated sequence reads derive from the same sequence comprising mutations may be based on the number of mutations with matching position λ_{x,y} and/or with mismatching position δ_{x,y}. In an embodiment, the measure correlated to the probability that the at least two mutated sequence reads derive from the same sequence comprising mutations corresponds to the number of mutations with matching position λ_{x,y}. The higher the number of mutations with matching positions λ_{x,y} is, the higher is the probability that the at least two mutated sequence reads derive from the same sequence comprising mutations. In an alternative embodiment, the measure correlated to the probability that the at least two mutated sequence reads derive from the same sequence comprising mutations corresponds to the number of mutations with mismatching position δ_{x,y}. The lower the number of mutations with mismatching positions δ_{x,y} is, the higher is the probability that the at least two mutated sequence reads derive from the same sequence comprising mutations.

In a preferred embodiment, the measure correlated to the probability that the at least two mutated sequence reads derive from the same sequence comprising mutations is one of i) a probability density that the at least two mutated sequence reads derive from the same sequence comprising mutations, and ii) a score function that is correlated to the probability density that the at least two mutated sequence reads derive from the same sequence comprising mutations.

For example, the number of mutations with matching positions λ_{x,y} and the number of mutations with mismatching positions δ_{x,y} can be used to compute a probability density under a model that the two reads are derived from the same sequence comprising mutations M, or a score function that is correlated with such a probability density. One such score function could be ω_{a,c}=Δ(λ_{x,y})−Δ(δ_{x,y}) where Δ(n)=(0.5n)(n+1), for a=b_{z,x}.i and c=b_{z,y}.i. In this way, ω_{a,c} represents the score or weight of a link between two mutated sequence reads ρ^{a} and ρ^{c}. A collection of such links can be produced for all pairs of mutated sequence reads ρⁱ in a respective minimizer bin b_{z}, or if there are a large number of entries in the minimizer bin b_{z} the link weight computation or reporting can be subsampled to randomly chosen pairs of entries in the minimizer bin b_{z}.

Step S300: Sequence Assembly or Sequence Reconstruction

The method 10 may further comprise a step S300 of assembling or reconstructing a sequence, or at least a portion of a sequence, for example a sequence comprising mutations or a sequence not comprising mutations. The assembled or reconstructed sequence may be a sequence comprising mutations or a sequence not comprising mutations.

The method 200, for example a step S300 of reconstructing or assembling a sequence, may comprise creating an undirected weighted graph from the plurality of mutated sequence reads. The undirected weighted graph comprises nodes corresponding to the plurality of mutated sequence reads. For example, each node may correspond to a respective mutated sequence read in that it is represented by the read index i of the respective mutated sequence read, or by the sequence of the respective mutated sequence read. The edges between the nodes are associated with respective edge weights, wherein each edge weight may be determined based on the number of mutations with matching position and/or with mismatching position determined for the two mutated sequence reads corresponding to the two nodes associated with the respective edge. Each edge weight may correspond to the measure correlated to the probability that the at least two mutated sequence reads (i.e. the two mutated sequence reads corresponding to the nodes associated with the edge associated with the edge weight) derive from the same sequence comprising mutations. As such, the edge weight connecting two mutated sequence reads (nodes) represents the probability that those two mutated sequence reads were derived from the same sequence comprising mutations, or some other arbitrary function that is correlated with that probability.

The undirected weighted graph can be constructed by processing each minimizer bin sequentially or in parallel, thereby computing edges among the mutated sequence reads in each minimizer bin. The edge weight may be the score function ω_{a,c}.

The undirected weighted graph including the edge weights ω_{a,c} may then be used in the processing of SAM data (e.g. mutated sequence reads), for example using any known or unknown techniques for using such an undirected weighted graph to assemble a sequence. Assembling a sequence from the undirected weighted graph may comprise, for example, creating clusters of mutated sequence reads, and assembling the mutated sequence reads in each cluster to reconstruct a template corresponding to at least a portion of the sequence comprising mutations.

For example, the method 200 or the step S300 of reconstructing or assembling at least a portion of a sequence may comprise performing graph clustering on the undirected weighted graph, thereby creating clusters of mutated sequence reads that are expected to derive from the same sequence comprising mutations. Graph clustering may be performed using any standard flow-based graph clustering algorithm, such as Markov clustering (MCL) or Infomap. Alternatively the edges of the undirected weighted graph could be filtered on some minimum weight threshold, and the connected components of the graph could then be taken to represent mutated sequence reads that derive from the same sequence comprising mutations.

The step S300 of reconstructing or assembling at least a portion of a sequence may further comprise reconstructing at least a portion of the sequence comprising mutations by assembling the mutated sequence reads in the clusters. For example, the mutated sequence reads in the clusters could be subjected to standard de novo assembly methods to reconstruct the sequence comprising mutations. Such de novo assembly methods include, for example, the IDBA-UD algorithm of “IDBA-UD: a de novo assembler for single-cell and metagenomic sequencing data with highly uneven depth”, Peng Y et al., Bioinformatics. 2012 Jun. 1; 28(11):1420-8. doi: 10.1093/bioinformatics/bts174. Epub 2012 Apr. 11, or the SPAdes method of “SPAdes: A New Genome Assembly Algorithm and Its Applications to Single-Cell Sequencing”, Benkevich A et al., J Comput Biol. 2012 May; 19(5): 455-477, or the A5-miseq method of “A5-miseq: an updated pipeline to assemble microbial genomes from Illumina MiSeq data”, Coil D et al, Bioinformatics. 2015 Feb. 15; 31(4):587-9. doi: 10.1093/bioinformatics/btu661. Epub 2014 Oct. 22.

The step S300 of reconstructing or assembling a sequence may further comprise reconstructing at least a portion of the sequence not comprising mutations using error correction on the reconstructed portion of the sequence comprising mutations, i.e. by inferring the most likely sequence not comprising mutations from the reconstructed portion of the sequence comprising mutations using the plurality of non-mutated sequence reads. Methods for such error correction include, for example, the FMLRC method of “FMLRC: Hybrid long read error correction using an FM-index”, Jeremy R. Wang et al., BMC Bioinformatics volume 19, Article number: 50 (2018). For example, the sequence comprising mutations could be subject to error correction using the non-mutated sequence reads to remove introduced mutations, thereby reconstructing portions of the sequence not comprising mutations. Error correction may comprise, for example, determining possible sets of edits to the sequence comprising mutations that would be required to transform the sequence comprising mutations into a sequence not comprising mutations that is compatible with the non-mutated sequence reads, determining the set of edits having the smallest size (i.e. comprising the least edits) from the possible sets of edits, and applying the determined set of edits having the smallest size to the sequence comprising mutations, thereby arriving a likely estimate of the sequence not comprising mutations. The portions of the sequence not comprising mutations could then be assembled using standard de novo long read assembly tools such as Canu, Flye, or PEREGRINE, or in hybrid with the short reads in R using a tool like Unicycler or MaSuRCA, thereby assembling the sequence not comprising mutations.

### Processing of Sample Pools

When processing sample batches comprising a plurality of samples, sample barcodes may be introduced in the form of defined sequence tags for each sample. If the user of the method 200 wishes to use the method on multiple samples, wherein each sample comprises one or more mutated target template nucleic acid molecules, one possibility is to process (e.g. mutate and/or fragment) each sample separately in the lab and then introduce sample barcodes only at the final step prior to sequencing. Another alternative is to introduce sample barcodes only at the ends of the target template nucleic acid molecules, in which case it becomes possible to pool all barcoded target template nucleic acid molecules early in the sample preparation process, thereby greatly reducing reagent and hands-on labour costs (the so-called early sample pooling approach). As such, sample preparation may comprise introducing respective sample barcodes at the ends of the target template nucleic acid molecules in each sample, such that each sample comprises target template nucleic acid molecules having a different sample barcode from the target template nucleic acid molecules in the other samples. Sample preparation may further comprise pooling the samples to create a sample pool, mutating and optionally fragmenting the target template nucleic acid molecules in the sample pool, and sequencing portions of the mutated target template nucleic acid molecules in the sample pool.

The early sample pooling approach introduces an additional challenge in the data processing, however, because the resulting plurality of mutated sequence reads P contains an unlabelled mix of mutated sequence P reads from many different samples. Samples may be processed separately to construct the non-mutated sequence reads R, in which case the non-mutated sequence reads R comprise a plurality of sets of non-mutated sequence reads R¹ . . . R_{ζ}, where ζ is the number of samples that have been processed in the batch. Each set of non-mutated sequence reads may be associated with a respective sample. The method 200 may comprise receiving the non-mutated sequence reads R in a plurality of sets of non-mutated sequence reads R¹ . . . R_{ζ} each set of non-mutated sequence reads R¹ . . . R_{ζ} being associated with a respective one or the plurality of samples.

Each of the plurality of mutated sequence reads may thus be a subsequence of a sequence comprising mutations associated with one of a plurality of samples. Each of the plurality of non-mutated sequence reads may correspond to a subsequence of a sequence not comprising mutations associated with the one of the plurality of samples. Each sequence comprising mutations may comprise mutations compared to a respective sequence not comprising mutations. Obtaining a set of non-mutated seed-masked k-mers may comprise obtaining a respective set of non-mutated seed-masked k-mers for each sample.

A simple approach to processing the data from the ζ samples would be to apply the method 200 once for each of the ζ samples. An alternative approach is to extend the method 200 such that all ζ samples can be processed concurrently. This may be achieved as described below.

The method 200 (e.g. step S230) may comprise creating a set of non-mutated sample bitvectors, each non-mutated sample bitvector defining, for a respective k-mer in the set of non-mutated seed-masked k-mers V_{R}, in which of the plurality of samples the respective k-mer exists (or exists at least x times, where x is an integer greater or equal to 1) and in which of the plurality of samples the respective k-mer does not exist (or exists less than x times). The set of non-mutated seed-masked k-mers V_{R} may be created from the plurality of non-mutated k-mers in the manner already described above. The plurality of non-mutated k-mers may be defined as the union of all k-mers in each of the plurality of samples R¹ . . . R_{ζ} i.e. the plurality of non-mutated k-mers R may be defined as R=∪R¹ . . . R_{ζ}.

For example, the method 200 may comprise defining a surjection of V_{R} onto a collection of bitvectors containing binary indicators for the presence of the seed-masked k-mers in each sample. Each bitvector may have a 1 in position i if the i-th sample in the plurality of samples contains the k-mer (or contains it at least X times), otherwise a 0 in position i. In a software implementation, the surjection could be stored using an unordered map data structure such as a hash map, or an approximate membership query structure such as a Counting Quotient Filter. The surjection may be denoted as Z: V_{R}→v where v is the space of bitvectors of length ζ.

The step S230 of determining positions of one or more mutations in each mutated sequence read may be extended to construct the bitvector α concurrently for multiple samples. Determining the positions of the one or more mutations may comprise, for each mutated sequence read, and for each set and/or each combination of sets of non-mutated seed-masked k-mers, identifying the one or more positions in the mutated sequence read that are masked by all of the seed patterns that correspond to the mutated seed-masked k-mers of the plurality of mutated seed-masked k-mers that exists in the respective set or combination of sets of non-mutated seed-masked k-mers, and associating the identified one or more positions with the one or more samples associated with the respective set or combination of sets of non-mutated seed-masked k-mers. This may be achieved, for example by a MultiSample variant morphomutsMS(ρⁱ,V_{R}) of the function morphomuts(ρⁱ,V_{R}) that comprises the steps of:
1. Initializing a set A of bitvectors α, with one initial bitvector a₀ of length 2 [symbol] and containing only 0s; initializing bitvector b of length 2k and containing only 1s; initializing a set C of bitvectors c, with one initial member c₀ of length ζ and containing only 1s; initializing a mapping Γ: C⇔A;
2. For each position j in the read between 1 and [symbol] -k:
   a. For each seed pattern ψ∈Ψ, determine ψ(k(ρⱼⁱ))
   b. If ψ(k(ρⱼⁱ))∈V_{R} perform the following steps:
      i. For each element of C (i.e. for each c∈C) compute d←c∧Z(ψ(k(ρⱼⁱ));
      ii. If d contains only 0s, then return to 2b.i to process the next element of C (or if no more exist, process the next seed pattern ψ or the next position j), otherwise continue with 2b.iii;
      iii. assign α←Γ(c)|(ψ(b)>>2j), where denotes bitwise logical OR and >> denotes the bit shift right operator, and remove c from C;
      iv. Add d to C and α to A and define the mapping d→a in Γ;
      v. If \acute{d}∧c is nonzero then add \acute{d}∧c to C and Γ(c) to A and define the mapping \acute{d}∧c→Γ(c) in Γ;
      vi. return to 2b.i to process the next element c of C. If no more c exist in C, return to 2a to process the next seed pattern ψ. If no more ψ exist in Ψ, return to 2 to process the next position j. Otherwise, continue with 3. otherwise:
3. Transform the bitvectors in A using the transformation applied to create a in the function morphomuts(•); and
4. Return C, A, and the mapping F as the result of the function.

Optionally, when too few positions (e.g. less than a predetermined number y, where y is an integer greater or equal to 1, preferably greater or equal to 2, 3, 4 or 5) have matched in a bitvector in A, the corresponding entries in C and A can be discarded. This is advantageous because such entries can occur due to random similarity among input samples and the resulting bitvectors are thus the result of erroneous matches to the wrong sample. By discarding these positions prior to further processing, unnecessary computation can be avoided. The method 200 may comprise comparing the number of identified positions to a predetermined number y, where y is an integer greater or equal to 1, preferably greater or equal to 2, and if the number of identified positions is less than the predetermined number y, discarding (or ignoring in further processing) the identified one or more positions and the association of the identified one or more position with the one or more samples.

The tuples that are stored in the minimizer bins can be extended to include the sample bitvector information in C. Specifically, the tuple stored may be the read index i, the position j of the minimizer in the mutated sequence read, and c and α, where c is the sample bitvector and α is the mutation bitvector as computed by the morphomutsMS(ρⁱ,V_{R}) function.

Subsequently when minimizer bins are processed to compute edge weights, each edge weight can be annotated with a corresponding sample set. If the bitwise logical AND of sample bitvectors associated with a pair of mutated sequence reads is zero, then the corresponding edge can be discarded. If the edge score is less than a predetermined threshold score, the edge can be discarded. When there are multiple possible edges between a pair of mutated sequence reads, it becomes possible to retain only the highest edge weight and the associated set of sample bitvectors for this edge can be computed as the bitwise logical AND among the sample bitvectors. This approach has the advantage that naturally occurring variation in the sequences of different samples can be distinguished from mutations introduced during the sample processing. The step S240 of counting the number of mutations with matching position and/or with mismatching position when the minimizers of two mutated sequence reads are aligned may be performed, for any pair of the one or more positions of the mutations identified for the two mutated sequence reads, only if there is an overlap in the samples associated with the respective pair of one or more positions of the mutations identified for the two mutated sequence reads, i.e. only if a pair of one or more positions of the mutations identified for the two mutated sequence reads is associated with at least one common sample.

If only the ends of the mutated target template nucleic acid molecules contain a sample tag, then some of the plurality of mutated sequence reads P will carry this sample tag. In particular, mutated sequence reads created from sequencing the ends of the mutated target template nucleic acid molecules will carry the sample tag. Following the clustering of mutated sequence reads it becomes possible to assign read clusters to samples simply by evaluating the presence of sample tagged reads in each cluster. When only a single sample tag appears in a cluster, assignment to a sample is straightforward and unambiguous. Performing graph clustering on the undirected weighted graph may comprise associating, to each cluster of mutated sequence reads, a sample tag comprised by at least one of the mutated sequence reads in the respective cluster.

Occasionally multiple sample tags may appear in a single cluster, either due to noise or error in the sequencing or data analysis procedures. In this case a confident sample assignment may still be possible if a large excess of one sample tag exists over other tags. In cases where a single assignment is not possible, it may still be possible to disambiguate the sample by using a semi-supervised graph decomposition procedure that decomposes the multi-sample cluster into a number of smaller clusters, with one cluster per sample tag. Even when a cluster contains no reads that carry a sample tag, it may still be possible to confidently assign the cluster to a sample, if the majority of the sample masks associated with the read links all indicate a single sample. Performing graph clustering on the undirected weighted graph may comprise identifying, in each cluster of mutated sequence reads, one or more sample tags comprised by the mutated sequence reads in the respective cluster of mutated sequence reads. Each cluster of mutated sequence reads may be associated with the sample tag that occurs most frequently in the mutated sequence reads in the respective cluster. Optionally, if two or more different sample tags are identified in a cluster of mutated sequence reads, the cluster of mutated sequence reads may be split into two or more clusters, each of the two or more clusters being associated with a respective one of the two or more different sample tags and containing different ones of the mutated sequence reads.

### Sample Preparation and Sequencing

The method 10 for determining at least a portion of a sequence of at least one target template nucleic acid molecule may comprise sequencing 100 regions of at least one target template nucleic acid molecule comprising mutations so as to provide the plurality of mutated sequence reads. The method 10 for determining at least a portion of a sequence of the at least one target template nucleic acid molecule may further comprise performing the method 200 of determining a measure correlated to the probability that two mutated sequence reads derive from the same sequence comprising mutations based on the plurality of mutated sequence reads obtained by the sequencing 100.

The step of sequencing may comprise:
a) providing a pair of samples, each sample comprising at least one target template nucleic acid molecule;
(b) sequencing regions of the at least one target template nucleic acid molecule in a first of the pair of samples to provide the plurality of non-mutated sequence reads;
(c) introducing mutations into the at least one target template nucleic acid molecule in a second of the pair of samples to provide the at least one mutated target template nucleic acid molecule;
(d) sequencing regions of the at least one mutated target template nucleic acid molecule to provide the plurality of mutated sequence reads.

In a preferred embodiment, the step of introducing mutations comprises introducing transition mutations into the at least one target template nucleic acid molecule in the second of the pair of samples.

The step of sequencing may comprise:
(a) providing a plurality of pairs of samples, each pair of samples comprising a first sample and a second sample, each sample comprising at least one target template nucleic acid molecule;
(b) introducing a sample barcode into the at least one target template nucleic acid molecule of each pair of samples, such that each pair of samples is associated with a respective sample barcode;
(c) sequencing regions of the at least one target template nucleic acid molecule in each first sample to provide the plurality of non-mutated sequence reads, wherein the sequencing is performed separately for each first sample, thereby providing a respective set of non-mutated sequence reads for each first sample;
(d) pooling the second samples, thereby creating a sample pool of second samples;
(e) introducing mutations into target template nucleic acid molecules in the sample pool to provide mutated target template nucleic acid molecules;
(d) sequencing regions of the mutated target template nucleic acid molecules to provide the plurality of mutated sequence reads.

Optionally the step of sequencing may further comprise fragmenting the at least one target template nucleic acid molecule in each first sample after introducing the sample barcode and before sequencing regions of the at least one target template nucleic acid molecule.

Optionally the step of sequencing may further comprise fragmenting the at least one target template nucleic acid molecule or the mutated target template nucleic acid molecules in the sample pool before sequencing regions of the mutated target template nucleic acid molecules.

In the methods of the invention, any number of at least one target template nucleic acid molecules may be sequenced simultaneously. Thus, in an embodiment of the invention, the at least one target template nucleic acid molecule comprises a plurality of target template nucleic acid molecules. Optionally, the at least one target template nucleic acid molecule comprises at least 10, at least 20, at least 50, at least 100, or at least 250 target template nucleic acid molecules. Optionally, the at least one target template nucleic acid molecule comprises between 10 and 1000, between 20 and 500, or between 50 and 100 target template nucleic acid molecules.

Step S110: Sample Preparation

Since the first of the pair of samples and the second of the pair of samples both comprise the at least one target template nucleic acid molecule, the pair of samples may be derived from the same target organism or taken from the same original sample.

For example, if the user intends to sequence the at least one target template nucleic acid molecule in a sample, the user may take a pair of samples from the same original sample. Optionally, the user may replicate the at least one target template nucleic acid molecule in the original sample before the pair of samples is taken from it. The user may intend to sequence various nucleic acid molecules from a particular organism, such as E. coli. If this is the case, the first of the pair of samples may be a sample of E. coli from one source, and the second of the pair of samples may be a sample of E. coli from a second source.

The pair of samples may originate from any source that comprises, or is suspected of comprising, the at least one target template nucleic acid molecule. The pair of samples may comprise a sample of nucleic acid molecules derived from a human, for example a sample extracted from a skin swab of a human patient. Alternatively, the pair of samples may be derived from other sources such as a water supply. Such samples could contain billions of template nucleic acid molecules. It would be possible to sequence each of these billions of target template nucleic acid molecules simultaneously using the methods of the invention, and so there is no upper limit on the number of target template nucleic acid molecules which could be used in the methods of the invention.

In an embodiment, multiple pairs of samples may be provided. For example, at least 2, 3, 4, 5, 6, 7, 8, 9, 10, 11, 15, 20, 25, 50, 75, 100, 500, 1000 or 5000 pairs of samples may be provided. Optionally, less than 10000, less than 5000, less than 1000, less than 100, less than 75, less than 50, less than 25, less than 20, less than 15, less than 11, less than 10, less than 9, less than 8, less than 7, less than 6, less than 5, or less than 4 samples are provided. Optionally, between 2 and 100, 2 and 75, 2 and 50, between 2 and 25, between 5 and 15, or between 7 and 15 pairs of samples are provided.

Where multiple pairs of samples are provided, the at least one target template nucleic acid molecules in different pairs of samples may be labelled with different sample tags (also referred to as sample barcodes herein). For example, if the user intends to provide 2 pairs of samples, all or substantially all of the at least one target template nucleic acid molecules in the first pair of samples may be labelled with sample tag A, and all or substantially all of the at least one target template nucleic acid molecules in the second pair of samples may be labelled with sample tag B.

Suitable methods for amplifying the at least one target template nucleic acid molecule are known in the art. For example, PCR is commonly used. PCR is described in more detail below under the heading “introducing mutations into the at least one target template nucleic acid molecule”.

Introducing Mutations into the at Least One Target Template Nucleic Acid Molecule

The method may comprise a step of introducing mutations into the at least one target template nucleic acid molecule in a second of the pair of samples to provide at least one mutated target template nucleic acid molecule.

The mutations may be substitution mutations, insertion mutations, or deletion mutations. For the purposes of the present invention, the term “substitution mutation” should be interpreted to mean that a nucleotide is replaced with a different nucleotide. For example, the conversion of the sequence ATCC to the sequence AGCC introduces a single substitution mutation. For the purposes of the present invention, the term “insertion mutation” should be interpreted to mean that at least one nucleotide is added to a sequence. For example, conversion of the sequence ATCC to the sequence ATTCC is an example of an insertion mutation (with an additional T nucleotide being inserted). For the purposes of the present invention, the term “deletion mutation” should be interpreted to mean that at least one nucleotide is removed from a sequence. For example, conversion of the sequence ATTCC to ATCC is an example of a deletion mutation (with a T nucleotide being removed). Preferably, the mutations are substitution mutations.

The phrase “introducing mutations into the at least one target template nucleic acid molecule” refers to exposing the at least one target template nucleic acid molecule in the second of the pair of samples to conditions in which the at least one target template nucleic acid molecule is mutated. This may be achieved using any suitable method. For example, mutations may be introduced by chemical mutagenesis and/or enzymatic mutagenesis.

Optionally, the step of introducing mutations into the at least one target template nucleic acid molecule mutates between 1% and 50%, between 3% and 25%, between 5% and 20%, or around 8% of the nucleotides of the at least one target template nucleic acid molecule. Optionally, the at least one mutated target template nucleic acid molecule comprises between 1% and 50%, between 3% and 25%, between 5% and 20%, or around 8% mutations.

The user can determine how many mutations are comprised within the at least one mutated target template nucleic acid molecule, and/or the extent to which the step of introducing mutations into the at least one target template nucleic acid molecule mutates the at least one target template nucleic acid molecule by performing the step of introducing mutations on a nucleic acid molecule of known sequence, sequencing the resultant nucleic acid molecule and determining the percentage of the total number of nucleotides that have changed compared to the original sequence.

Optionally, the step of introducing mutations into the at least one target template nucleic acid molecule mutates the at least one target template nucleic acid molecule in a substantially random manner. Optionally, the at least one mutated target template nucleic acid molecule comprises a substantially random mutation pattern.

The at least one mutated target template nucleic acid molecule comprises a substantially random mutation pattern if it contains mutations throughout its length at substantially similar levels. For example, the user can determine whether the at least one mutated target template nucleic acid molecule comprises a substantially random mutation pattern by mutating a test nucleic acid molecule of known sequence to provide a mutated test nucleic acid molecule. The sequence of the mutated test nucleic acid molecule may be compared to the test nucleic acid molecule to determine the positions of each of the mutations. The user may then determine whether the mutations occur throughout the length of the mutated test nucleic acid molecule at substantially similar levels by:
(i) calculating the distance between each of the mutations;
(ii) calculating the mean of the distances;
(iii) sub-sampling the distances without replacement to a smaller number such as 500 or 1000;
(iv) constructing a simulated set of 500 or 1000 distances from the geometric distribution, with a mean given by the method of moments to match that previously computed on the observed distances; and
(v) computing a Kolmolgorov-Smirnov on the two distributions.

The at least one mutated target template nucleic acid molecule may be considered to comprise a substantially random mutation pattern if D<0.15, D<0.2, D<0.25, or D<0.3, depending on the length of the non-mutated reads.

Similarly, the step of introducing mutations into the at least one target template nucleic acid molecule mutates the at least one target template nucleic acid molecule in a substantially random manner, if the resultant at least one mutated target template nucleic acid molecule comprises a substantially random mutation pattern. Whether a step of introducing mutations into the at least one target template nucleic acid molecule does mutate the at least one target template nucleic acid molecule in a substantially random manner may be determined by carrying out the step of introducing mutations into the at least one target template nucleic acid molecule on a test nucleic acid molecule of known sequence to provide a mutated test nucleic acid molecule. The user may then sequence the mutated test nucleic acid molecule to identify which mutations have been introduced and determine whether the mutated test nucleic acid molecule comprises a substantially random mutation pattern.

Optionally, the at least one mutated target template nucleic acid molecule comprises an unbiased mutation pattern. Optionally, the step of introducing mutations into the at least one target template nucleic acid molecule introduces mutations in an unbiased manner. The at least one mutated target template nucleic acid molecule comprises an unbiased mutation pattern, if the types of mutations that are introduced are random. If the mutations that are introduced are substitution mutations, then the mutations that are introduced are random if a similar proportion of A (adenosine), T (thymine), C (cytosine) and G (guanine) nucleotides are introduced. By the phrase “a similar proportion of A (adenosine), T (thymine), C (cytosine) and G (guanine) nucleotides are introduced”, we mean that the number of adenosine, the number of thymine, the number of cytosine and the number of guanine nucleotides that are introduced are within 20% of one another (for example 20 A nucleotides, 18 T nucleotides, 24 C nucleotides and 22 G nucleotides could be introduced).

Whether a step of introducing mutations into the at least one target template nucleic acid molecule does mutate the at least one target template nucleic acid molecule in a unbiased manner may be determined by carrying out the step of introducing mutations into the at least one target template nucleic acid molecule on a test nucleic acid molecule of known sequence to provide a mutated test nucleic acid molecule. The user may then sequence the mutated test nucleic acid molecule to identify which mutations have been introduced and determine whether the mutated test nucleic acid molecule comprises an unbiased mutation pattern.

Usefully, the methods of generating a sequence of at least one target template nucleic acid molecule may be used even when the step of introducing mutations into the at least one target template nucleic acid molecule introduces unevenly distributed mutations. Thus, in one embodiment the at least one mutated target template nucleic acid molecule comprises unevenly distributed mutations. Optionally, the step of introducing mutations into the at least one mutated target template nucleic acid molecule introduces mutations that are unevenly distributed. Mutations are considered to be “unevenly distributed” if the mutations are introduced in a biased manner, i.e. the number of adenosine, the number of thymine, the number of cytosine, and the number of guanine nucleotides that are introduced are not within 20% of one another. Whether the at least one mutated target template nucleic acid molecule comprises unevenly distributed mutations, or the step of introducing mutations into the at least one target template nucleic acid molecule introduces mutations that are unevenly distributed may be determined in a similar way to that described above for determining whether the step of introducing mutations into the at least one target template nucleic acid molecule introduces mutations in an unbiased manner.

Similarly, the methods of generating a sequence of at least one target template nucleic acid molecule may be used even when the mutated sequence reads and/or the non-mutated sequence reads comprise unevenly distributed sequencing errors. Thus, in one embodiment, the mutated sequence reads and/or the non-mutated sequence reads comprise sequencing errors that are unevenly distributed. Similarly, in one embodiment, the step of sequencing regions of the at least one target template nucleic acid molecule and/or the sequencing regions of the at least one mutated target template nucleic acid molecule introduces sequence errors that are unevenly distributed.

Whether a particular step of sequencing regions of the at least one target template nucleic acid molecule and/or sequencing regions of the at least one mutated target template nucleic acid molecule introduces sequence errors that are unevenly distributed will likely depend on the accuracy of the sequencing instrument and will likely be known to the user. However, the user may investigate whether a step of sequencing regions of the at least one target template nucleic acid molecule and/or the sequencing regions of the at least one mutated target template nucleic acid molecule introduces sequence errors that are unevenly distributed by performing the sequencing method on a nucleic acid molecule of known sequence and comparing the sequence reads produced with those of the original nucleic acid molecule of known sequence. The user may then apply the probability function discussed in Example 6, and determine values for M and E. If the values of the E and the matrix model are unequal or substantially unequal (within 10% of one another), then the step of sequencing regions of the at least one target template nucleic acid molecule introduces sequence errors that are unevenly distributed.

Introducing mutations into the at least one target template nucleic acid molecule via chemical mutagenesis may be achieved by exposing the at least one target template nucleic acid to a chemical mutagen. Suitable chemical mutagens include Mitomycin C (MMC), N-methyl-N-nitrosourea (MNU), nitrous acid (NA), diepoxybutane (DEB), 1,2,7,8,-diepoxyoctane (DEO), ethyl methane sulfonate (EMS), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamino)-acridinedihydrochloride (ICR-170), 2-amino purine (2A), bisulphite, and hydroxylamine (HA). For example, when nucleic acid molecules are exposed to bisulphite, the bisulphite deaminates cytosine to form uracil, effectively introducing a C-T substitution mutation.

As noted above, the step of introducing mutations into the at least one target template nucleic acid molecule may be carried out by enzymatic mutagenesis. Optionally, the enzymatic mutagenesis is carried out using a DNA polymerase. For example, some DNA polymerases are error-prone (are low fidelity polymerases) and replicating the at least one target template nucleic acid molecule using an error-prone DNA polymerase will introduce mutations. Taq polymerase is an example of a low fidelity polymerase, and the step of introducing mutations into the at least one target template nucleic acid molecule may be carried out by replicating the at least one target template nucleic acid molecule using Taq polymerase, for example by PCR.

The DNA polymerase may be a low bias DNA polymerase.

If the step of introducing mutations into the at least one target template nucleic acid molecule is carried out using a DNA polymerase, the at least one target template nucleic acid molecule may be incubated with the DNA polymerase and suitable primers under conditions suitable for the DNA polymerase to catalyse the generation of at least one mutated target template nucleic acid molecule.

Suitable primers comprise short nucleic acid molecules complementary to regions flanking the at least one target template nucleic acid molecule or to regions flanking nucleic acid molecules that are complementary to the at least one target template nucleic acid molecule. For example, if the at least one target template nucleic acid molecule is part of a chromosome, the primers will be complementary to regions of the chromosome immediately 3′ to the 3′ end of the at least one target template nucleic acid molecule and immediately 5′ to the 5′ end of the at least one target template nucleic acid molecule, or the primers will be complementary to regions of the chromosome immediately 3′ to the 3′ end of a nucleic acid molecule complementary to the at least one target template nucleic acid molecule and immediately 5′ to the 5′ end of a nucleic acid molecule complementary to the at least one target template nucleic acid molecule.

Suitable conditions include a temperature at which the DNA polymerase can replicate the at least one target template nucleic acid molecule. For example, a temperature of between 40° C. and 90° C., between 50° C. and 80° C., between 60° C. and 70° C., or around 68° C.

The step of introducing mutations into the at least one template nucleic acid molecule may comprise multiple rounds of replication. For example, the step of introducing mutations into the at least one target template nucleic acid molecule preferably comprises:
i) a round of replicating the at least one target template nucleic acid molecule to provide at least one nucleic acid molecule that is complementary to the at least one target template nucleic acid molecule; and
ii) a round of replicating the at least one target template nucleic acid molecule to provide replicates of the at least one target template nucleic acid molecule.

Optionally, the step of introducing mutations into the at least one target template nucleic acid molecule comprises at least 2, at least 4, at least 6, at least 8, at least 10, less than 10, less than 8, around 6, between 2 and 8, or between 1 and 7 rounds of replicating the at least one target template nucleic acid molecule. The user may choose to use a low number of rounds of replication to reduce the possibility of introducing amplification bias.

Optionally, the step of introducing mutations into the at least one target template nucleic acid molecule comprises at least 2, at least 4, at least 6, at least 8, at least 10, less than 10, less than 8, around 6, between 2 and 8, or between 1 and 7 rounds of replication at a temperature between 60° C. and 80° C.

Optionally, the step of introducing mutations into the at least one target template nucleic acid molecule is carried out using the polymerase chain reaction (PCR). PCR is a process that involves multiple rounds of the following steps for replicating a nucleic acid molecule:
a) melting;
b) annealing; and
c) extension and elongation.

The nucleic acid molecule (such as the at least one target template nucleic acid molecule) is mixed with suitable primers and a polymerase. In the melting step, the nucleic acid molecule is heated to a temperature above 90° C. such that a double-stranded nucleic acid molecule will denature (separate into two strands). In the annealing step, the nucleic acid molecule is cooled to a temperature below 75° C., for example between 55° C. and 70° C., around 55° C., or around 68° C., to allow the primers to anneal to the nucleic acid molecule. In the extension and elongation steps, the nucleic acid molecule is heated to a temperature greater than 60° C. to allow the DNA polymerase to catalyse primer extension, the addition of nucleotides complementary to the template strand.

Optionally, the step of introducing mutations into the at least one target template nucleic acid molecule comprises replicating the at least one target template nucleic acid molecule using Taq polymerase, in error-prone reactions conditions. For example, the step of introducing mutations into the at least one target template nucleic acid molecule may comprise PCR using Taq polymerase in the presence of Mn²⁺, Mg²⁺ or unequal dNTP concentrations (for example an excess of cytosine, guanine, adenine or thymine).

Step S120: Sequencing

### Obtaining Data Comprising Non-Mutated Sequence Reads and Mutated Sequence Reads

The methods of the invention may comprise a step of receiving mutated sequence reads, and optionally receiving non-mutated sequence reads. The non-mutated sequence reads and the mutated sequence reads may be obtained from any source.

Optionally, the non-mutated sequence reads are obtained by sequencing regions of at least one target template nucleic acid molecule in a first of a pair of samples. Optionally, the mutated sequence reads are obtained by introducing mutations into the at least one target template nucleic acid molecule in a second of the pair of samples to provide at least one mutated target template nucleic acid molecule, and sequencing regions of the at least one mutated target template nucleic acid molecule.

Optionally, the non-mutated sequence reads comprise sequences of regions of at least one target template nucleic acid molecule in a first of a pair of samples, the mutated sequence reads comprise sequences of regions of at least one mutated target template nucleic acid molecule in a second of a pair of samples, and the pair of samples were taken from the same original sample or are derived from the same organism.

### Sequencing Regions of the at Least One Target Template Nucleic Acid Molecule or the at Least One Mutated Target Template Nucleic Acid Molecule

The method for determining a sequence of at least one target template nucleic acid molecule may comprise a step of sequencing regions of the at least one target template nucleic acid molecule in a first of the pair of samples to provide non-mutated sequence reads and/or a step of sequencing regions of the at least one mutated target template nucleic acid molecule to provide mutated sequence reads.

The sequencing steps may be carried out using any method of sequencing. Examples of possible sequencing methods include Maxam Gilbert Sequencing, Sanger Sequencing, sequencing comprising bridge amplification (such as bridge PCR), or any high throughput sequencing (HTS) method as described in Maxam A M, Gilbert W (February 1977), “A new method for sequencing DNA”, Proc. Natl. Acad. Sci. U.S.A 74 (2): 560-4, Sanger F, Coulson A R (May 1975), “A rapid method for determining sequences in DNA by primed synthesis with DNA polymerase”, J. Mol. Biol. 94 (3): 441-8; and Bentley D R, Balasubramanian S, et al. (2008), “Accurate whole human genome sequencing using reversible terminator chemistry”, Nature, 456 (7218): 53-59.

In a typical embodiment at least one, or preferably both, of the sequencing steps involve bridge amplification. Optionally, the bridge amplification step is carried out using an extension time of greater than 5, greater than 10, greater than 15, or greater than 20 seconds. An example of the use of bridge amplification is in Illumina Genome Analyzer Sequencers. Preferably paired-end sequencing is used.

Optionally, steps (i) of sequencing regions of the at least one target template nucleic acid molecule in a first of the pair of samples to provide non-mutated sequence reads and (ii) of sequencing regions of the at least one mutated target template nucleic acid molecule to provide mutated sequence reads are carried out using the same sequencing method.

Optionally steps (i) of sequencing regions of the at least one target template nucleic acid molecule in a first of the pair of samples to provide non-mutated sequence reads and (ii) of sequencing regions of the at least one mutated target template nucleic acid molecule to provide mutated sequence reads are carried out using different sequencing methods.

Optionally, steps (i) of sequencing regions of the at least one target template nucleic acid molecule in a first of the pair of samples to provide non-mutated sequence reads and (ii) of sequencing regions of the at least one mutated target template nucleic acid molecule to provide mutated sequence reads may be carried out using more than one sequencing method. For example, a fraction of the at least one target template nucleic acid molecules in the first of the pair of samples may be sequenced using a first sequencing method, and a fraction of the at least one target template nucleic acid molecules in the first of the pair of samples may be sequenced using a second sequencing method. Similarly, a fraction of the at least one mutated target template nucleic acid molecules may be sequenced using a first sequencing method, and a fraction of the at least one mutated target template nucleic acid molecules may be sequenced using a second sequencing method.

Optionally, steps (i) of sequencing regions of the at least one target template nucleic acid molecule in a first of the pair of samples to provide non-mutated sequence reads and (ii) of sequencing regions of the at least one mutated target template nucleic acid molecule to provide mutated sequence reads are carried out at different times. Alternatively, steps (i) and (ii) may be carried out fairly contemporaneously, such as within 1 year of one another. The first of the pair of samples and the second of the pair of samples need not be taken at the same time as one another. Where the two samples are derived from the same organism, they may be provided at substantially different times, even years apart, and so the two sequencing steps may also be separated by a number of years. Furthermore, even if the first of the pair of samples and the second of the pair of samples were derived from the same original sample, biological samples can be stored for some time and so there is no need for the sequencing steps to take place at the same time.

The mutated sequence reads and/or the non-mutated sequence reads may be single ended or paired-ended sequence reads.

Optionally, the mutated sequence reads and/or the non-mutated sequence reads are greater than 50 nt, greater than 100 nt, greater than 500 nt, less than 200,000 nt, less than 15,000 nt, less than 1,000 nt, between 50 and 200,000 nt, between 50 and 15,000 nt, or between 50 and 1,000 nt.

Optionally, the sequencing steps are carried out using a sequencing depth of between 0.1 and 500 reads, between 0.2 and 300 reads, or between 0.5 and 150 reads per nucleotide per at least one target template nucleic acid molecule. The greater the sequencing depth, the greater the accuracy of the sequence that is determined/generated will be, but assembly may be more difficult.

### Choice of Parameters

Preferably, the parameters used in the method 200 are chosen as set out below.

In a preferred embodiment, the weight w(ψ) of each seed pattern ψ is in the range from 5 to 50, preferably from 10 to 30, further preferable from 13 to 23. This ensures that each seed pattern ψ is large enough to ensure that each k-mer masked by each seed pattern ψ is unique with high probability. For example, for bacterial genomes with a typical length of 5 million nucleotides, the weight w(ψ) of each seed pattern ψ is preferably in the range of 13-19, noting that that 4¹³>5 million. For human-size genomes with typical lengths around 3 billion nucleotides, the weight w(ψ) of each seed pattern ψ is preferably in the range of 19-23, noting that 4¹⁹>3×10⁹.

In a preferred embodiment, the size k of each k-mer used in the step S230 of determining the positions of the one or more mutations in each mutated sequence read is greater than weight w(ψ) of each seed pattern ψ. The size k of each k-mer may be less than 5 times, less than 4 times, less than 3 times, or less than 2 times the weight w(ψ) of each seed pattern ψ. The size k of each k-mer used in the step S230 of determining the positions of the one or more mutations in each mutated sequence read may be in the range from 10 to 250, preferably from 13 to 100, further preferably from 15 to 50, most preferably from 20 to 40. This ensures that the size k is small enough to ensure that the probability that any k-mer includes an insertion or deletion sequencing error, which is disadvantageous in the context of the method 200, is low.

An example seed family Ψ comprising seed patterns ψ with weight w(ψ)=16 and k=27 is shown below:

ψ₁={0, 1, 2, 3, 5, 6, 9, 12, 13, 14, 16, 18, 20, 21, 22, 23},

ψ₂={0, 1, 2, 4, 5, 9, 10, 11, 13, 18, 19, 21, 23, 24, 25, 26},

ψ₃={0, 1, 2, 3, 4, 5, 7, 8, 9, 10, 13, 15, 16, 18, 19, 20},

«₄={0, 1, 2, 4, 6, 7, 12, 14, 16, 17, 20, 21, 23, 24, 25, 26},

In an embodiment, the k-mers used in the step S220 of applying a common minimizer function, i.e. the one or more minimizers determined for each mutated sequence read, have a size k different from the k-mers used in the step S230 of determining the positions of the one or more mutations in each mutated sequence read. The size k of each minimizer may be in the range from 5 to 50, preferably from 10 to 30, further preferable from 13 to 23. The size k of each minimizer may be chosen based on the same considerations as in the choice of the weight w(ψ) of the seed patterns. The size k of each minimizer may be in the range from 13 to 19 for bacteria and from 19 to 23 for human-sized genomes.

### Implementation of the Method 200

The method 200 can be implemented in a variety of ways. A preferred approach is to first compute the set U_{M} in an initial pass through some or all of the mutated sequence reads P and non-mutated sequence reads R, then to compute W_{M} in a second pass through the mutated sequence reads P and non-mutated sequence reads R. With W_{M} in hand, in a third pass through the mutated sequence reads P the minimizer positions can be computed along with the positions of the one or more mutations, and these can be stored in the minimizer bins either in RAM or fixed storage (e.g. disk). Optionally multiple minimizer bins can be stored in a single file, either sorted or unordered. Then each minimizer bin (or each file) can be read sequentially or in parallel, with the minimizer bins processed to compute the edge weights. Because each mutated sequence read can appear in multiple minimizer bins it is possible for a pair of mutated sequence reads to have multiple estimates of their weight computed. When this occurs some measure must be used to select a preferred weight, usually the maximum. Finally, when the sequencing chemistry has produced paired-end reads, and each in a pair of paired-end reads share common minimizers, then the scores of the two ends can be summed to arrive at a single score for the pair of paired-end reads.

### Experimental Data

The method 200 was used to process several real SAM datasets, each SAM dataset comprising non-mutated sequence reads and mutated sequence reads.

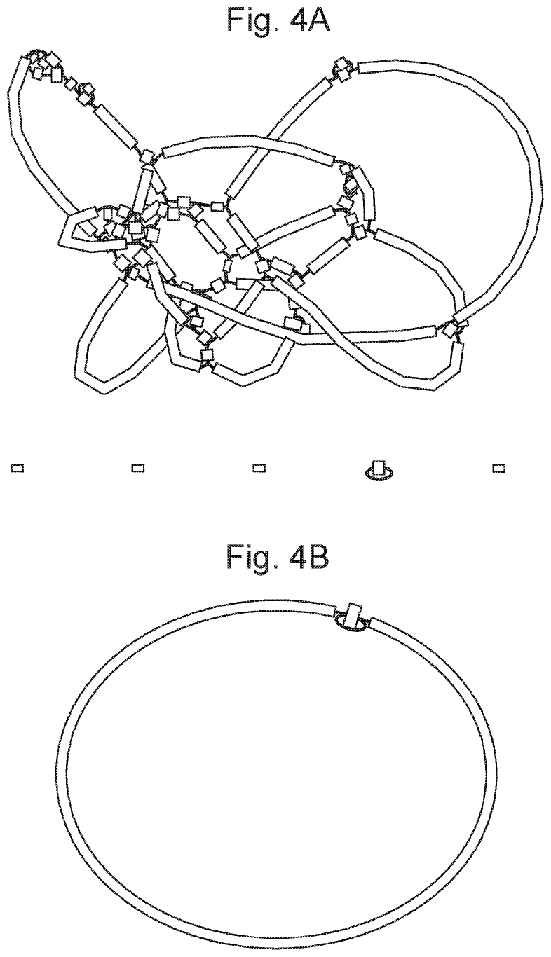

A SAM dataset of Arcobacter butzleri JV22 was processed. This organism has a 2.3 Mbp genome that exists as a single circular chromosome. A C++ implementation of the method 200 was run on an Amazon AWS instance. The SAM dataset consists of 956133 reference read pairs (unmutated) and 2154909 mutated read pairs derived from approximately 8000 mutated long templates. 2087506 of the mutated reads (96.9%) derive from internal parts of the mutated long templates while 67403 (3.1%) derive from the ends of long templates and contain sample barcodes. Each individual read is of length 150 nt or less. The read pairs had previously been quality and adapter trimmed. The method 200 required 12 minutes CPU time and 1.2 GB RAM to process the dataset, producing 30033939 candidate links among reads. These links were then subjected to graph clustering using Markov Clustering (mcl), and the resulting 6779 groups of reads were de novo assembled using MEGAHIT (see Dinghua Li, Chi-Man Liu, Ruibang Luo, Kunihiko Sadakane, and Tak-Wah Lam. MEGAHIT: an ultra-fast single-node solution for large and complex metagenomics assembly via succinct de Bruijn graph. Bioinformatics, Oxford, England, 31(10):1674{1676, May 2015) to produce reconstructions of long mutated templates. Finally, the long mutated templates were used together with the unmutated reads in a hybrid genome assembly computed by the Unicycler software. The resulting assembly is shown in FIG. 4B, as compared to the assembly obtained from short reads alone (shown in FIG. 4A). FIG. 4A shows short read assembly of the 2.3 Mbp Arcobacter butzlerii genome using the Shovill assembly pipeline, prior to performing the method 200. This yielded an assembly in 78 scaffolds, with the largest scaffold covering 342 kbp and a scaffold N50 of about 127 kbp. FIG. 4B shows assembly of the 2.3 Mbp Arcobacter butzlerii genome using the method 200. The circular chromosome has largely resolved to a single contig, with the copy number of a small piece <200 nt remaining unresolved.

The scalability and resolving power of the approach implemented in the method 200 was measured using simulated data. A 50 kbp sequence from the CFTR gene was used to simulate increasing amounts of mutated long template coverage and corresponding mutated short reads from these templates. The simulations were carried out using newly developed scripts that first generate long mutated templates, then invoke the well-known Illumina read simulator artsim to simulate short read sequencing from the mutated templates. In addition to mutated data, 30× coverage of the unmutated sequence was simulated with artsim. We simulated long mutated template coverage ranging from 10¹ to 10⁶ in order-of-magnitude increments. The mutation rate was fixed at 6%. For each long template, 10× short read coverage was simulated. Results on the simulated data were evaluated by measuring the rate at which false positive links were reported by the method 200.

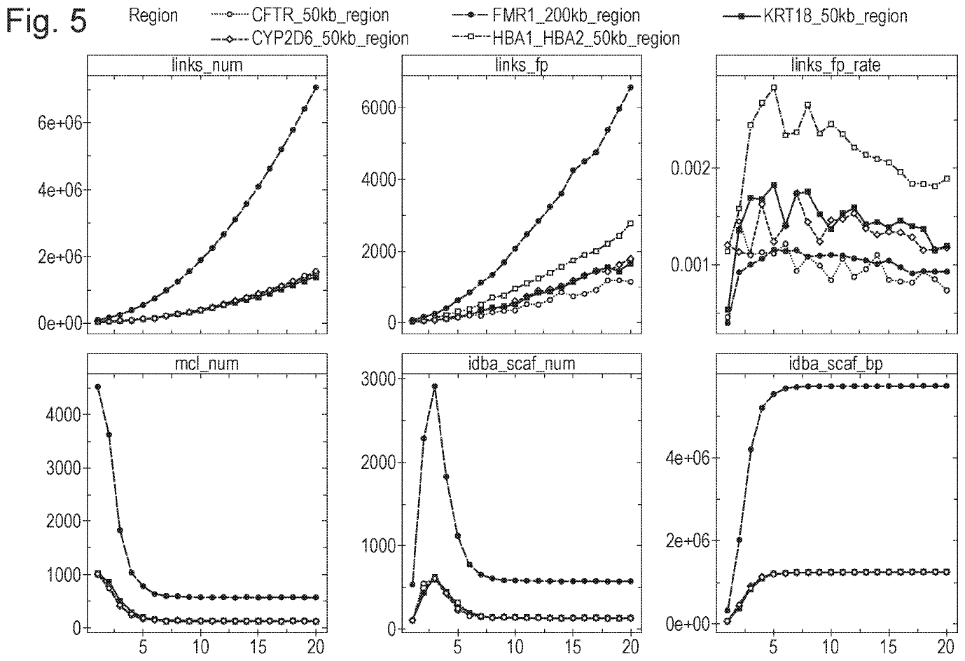

FIG. 5 shows the effect of depth of short read coverage per long template. The amount of short-read data generated per long template is shown on the x-axis, with the y-axis showing various performance metrics on results from the method 200. It can be seen that when short read coverage per template is low, e.g. <4×, poor and incomplete reconstructions of the original long templates is obtained. However when coverage per mutated template is in the range of 5-10×, good reconstructions can be obtained.
links num: number of links among mutated reads reported by the method 200. links fp: number of false positive links reported.
links fp rate: fraction of links that are false positive out of all reported links.
mcl num: number of clusters produced by Markov clustering on the graph reported by mmdreaming.
idba scaf num: number of scaffold sequences reconstructed by assembling the clusters of mutated short reads.
idba scaf bp: the sum of the lengths of all scaffolds assembled.

1. A computer-implemented method for determining a measure correlated to the probability that two mutated sequence reads derive from the same sequence comprising mutations, the method comprising:
receiving a plurality of mutated sequence reads, each mutated sequence read corresponding to a subsequence of a sequence comprising mutations, wherein the sequence comprising mutations comprises mutations compared to a sequence not comprising mutations;
applying a common minimizer function to each mutated sequence read, thereby determining one or more respective minimizers for each mutated sequence read;
determining positions of the one or more respective minimizers in each mutated sequence read;
determining positions of one or more mutations in each mutated sequence read; and for at least two mutated sequence reads with a common minimizer, counting the number of mutations with matching position and/or with mismatching position when the respective minimizers are aligned.

2. The method of claim 1, further comprising receiving a plurality of non-mutated sequence reads, each non-mutated sequence read corresponding to a subsequence of the sequence not comprising mutations.

3. The method of claim 1 or 2, wherein the step of applying a common minimizer function to each mutated sequence read comprises identifying i) the one or more k-mers in the respective mutated sequence read that is or are listed first in an ordered list of possible k-mers, or ii) the one or more k-mers that exist in a predetermined set of possible k-mers, wherein the one or more minimizers determined for the respective mutated sequence read are the identified one or more k-mers.

4. The method of claim 3, wherein, i) in the ordered list of possible k-mers, the k-mers are ordered based on the probability that the k-mers exist in the sequence comprising mutations and do not exist in the sequence not comprising mutations, or ii) the predetermined set of possible k-mers comprises k-mers that are relatively likely to exist in the sequence comprising mutations and not in the sequence not comprising mutations, optionally wherein
the predetermined set of possible k-mers does not comprise k-mers that are relatively unlikely to exist in the sequence comprising mutations.

5. The method of claim 2 and claim 3 or 4, wherein the ordered list of possible k-mers or the predetermined set of possible k-mers consists of k-mers that exist more often in the plurality of mutated sequence reads than in the plurality of non-mutated sequence reads, optionally wherein k-mers that exist more often in the plurality of mutated sequence reads than in the plurality of non-mutated sequence reads are relatively likely to exist in the sequence comprising mutations.

6. The method of claim 2 and any one of claims 3 to 5, wherein the predetermined set of possible k-mers consists of k-mers that exist n or more times in the plurality of mutated sequence reads and exist less than n times in the plurality of non-mutated sequence reads, where n is an integer greater or equal to 1, optionally wherein k-mers that exist n or more times in the plurality of mutated sequence reads and exist less than n times in the plurality of non-mutated sequence reads are relatively likely to exist in the sequence comprising mutations.

7. The method of claim 6, wherein the predetermined set of possible k-mers consists of k-mers that do not exist in the plurality of non-mutated sequence reads.

8. The method of claim 6 or 7, wherein n is 2.

9. The method of claim 2 and any one of claims 3 to 8, further comprising generating the ordered list of possible k-mers or the predetermined set of possible k-mers based on a comparison of the k-mers in the plurality of mutated sequence reads and the k-mers in the plurality of non-mutated sequence reads.

10. The method of any one of the preceding claims, wherein each minimizer is a k-mer of length greater than 5, preferably greater than 10.

11. The method of any one of the preceding claims, further comprising binning the mutated sequence reads in one or more minimizer bins, such that each minimizer bin contains mutated sequence reads having a common minimizer and does not contain mutated sequence reads not having a common minimizer, and
wherein the step of counting the number of mutations with matching position and/or with mismatching position is performed only on mutated sequence reads in the same minimizer bin.

12. The method of claim 2 and any one of claims 2 to 11, wherein the step of determining the positions of the one or more mutations in each mutated sequence read comprises:
obtaining a set of non-mutated seed-masked k-mers by applying each one of one or more seed patterns to k-mers in the plurality of non-mutated sequence reads;
for each mutated sequence read, applying the one or more seed patterns to k-mers in the respective mutated sequence read to obtain a plurality of mutated seed-masked k-mers, and determining the positions of the one or more mutations by identifying the one or more positions in the mutated sequence read that are masked by all of the seed patterns that correspond to the mutated seed-masked k-mers of the plurality of mutated seed-masked k-mers that exists in the set of non-mutated seed-masked k-mers.

13. The method of claim 12, wherein the one or more seed patterns are chosen such that the probability of obtaining identical seed-masked k-mers by applying at least one of the one or more seed patterns to any k-mer in the plurality of mutated sequence reads and to a corresponding k-mer in the plurality of non-mutated sequence reads is greater than 90%, preferably greater than 99%.

14. The method of claim 12 or 13, wherein the sequence comprising mutations comprises transition mutations compared to the sequence not comprising mutations; and
wherein the one or more seed patterns consist of one or more transition seed patterns.

15. The method of any one of claims 12 to 14, wherein each of the plurality of mutated sequence reads corresponds to a subsequence of a sequences comprising mutations associated with one of a plurality of samples, and each of the plurality of non-mutated sequence reads corresponds to a subsequence of a sequence not comprising mutations associated with the one of the plurality of samples, wherein each sequence comprising mutations comprises mutations compared to a respective sequence not comprising mutations;
wherein obtaining a set of non-mutated seed-masked k-mers comprises obtaining a respective set of non-mutated seed-masked k-mers for each sample;
further comprising creating a set of non-mutated sample bitvectors, each non-mutated sample bitvector defining, for a respective k-mer in the set of non-mutated seed-masked k-mers, in which of the plurality of samples the respective k-mer exists; and
wherein determining the positions of the one or more mutations comprises, for each mutated sequence read, and for each set and/or each combination of sets of non-mutated seed-masked k-mers, identifying the one or more positions in the mutated sequence read that are masked by all of the seed patterns that correspond to the mutated seed-masked k-mers of the plurality of mutated seed-masked k-mers that exists in the respective set or combination of sets of non-mutated seed-masked k-mers, and associating the identified one or more positions with the one or more samples associated with the respective set or combination of sets of non-mutated seed-masked k-mers.

16. The method of any one of claims 2 to 15, wherein the step of determining the positions of the one or more mutations in each mutated sequence read comprises:
for one or more of the mutated sequence reads, aligning the respective mutated sequence read to a reference assembly; and
determining the positions of the one or more mutations in the respective mutated sequence read by identifying positions in the respective mutated sequence read of differences between the respective mutated sequence read and the reference assembly.

17. The method of claim 16 when dependent on any one of claims 12 to 15, wherein the step of determining the positions of the one or more mutations in each mutated sequence read comprises for each mutated sequence read:
if a position in the respective mutated sequence read is aligned to the reference assembly, determining the position in the respective mutated sequence read to be a position of a mutation in the respective mutated sequence read if the position in the respective mutated sequence read is a position at which the respective mutated sequence read differs from the reference assembly; and
if a position in the respective mutated sequence read is not aligned to the reference assembly, determining the position in the respective mutated sequence read to be a position of a mutation in the respective mutated sequence read if the position in the respective mutated sequence read is a position that is masked by all of the seed patterns that correspond to the mutated seed-masked k-mers of the plurality of mutated seed-masked k-mers that exists in the set of non-mutated seed-masked k-mers.

18. The method of any one of the preceding claims, comprising determining the measure correlated to the probability that the at least two mutated sequence reads derive from the same sequence comprising mutations based on the number of mutations with matching position and/or with mismatching position.

19. The method of claim 18, wherein the measure correlated to the probability that the at least two mutated sequence reads derive from the same sequence comprising mutations is one of i) a probability density that the at least two mutated sequence reads derive from the same sequence comprising mutations, and ii) a score function that is correlated to the probability density that the at least two mutated sequence reads derive from the same sequence comprising mutations.

20. The method of any one of the preceding claims, further comprising creating an undirected weighted graph from the plurality of mutated sequence reads,
wherein the undirected weighted graph comprises nodes corresponding to the plurality of mutated sequence reads, and wherein the edges between the nodes are associated with respective edge weights, wherein each edge weight is determined based on the number of mutations with matching position and/or with mismatching position determined for the two mutated sequence reads corresponding to the two nodes associated with the respective edge.

21. The method of claim 20, wherein the edge weights correspond to the measure correlated to the probability that the at least two mutated sequence reads corresponding to the two nodes associated with the respective edge derive from the same sequence comprising mutations.

22. The method of claim 20 or 21, further comprising performing graph clustering on the undirected weighted graph, thereby creating clusters of mutated sequence reads that are expected to derive from the same sequence comprising mutations.

23. The method of claim 22, wherein the graph clustering comprises Markov clustering or Infomap.

24. The method of claim 22 or 23, further comprising reconstructing at least a portion of the sequence comprising mutations by assembling the mutated sequence reads in the clusters.

25. The method of claims 2 and 24, further comprising reconstructing at least a portion of the sequence not comprising mutations by inferring at least a portion of the likely sequence not comprising mutations from the reconstructed portion of the sequence comprising mutations, optionally using the plurality of non-mutated sequence reads.

26. A method for generating at least a portion of a sequence of a target template nucleic acid molecule, comprising the method of any one of claims 20 to 25.

27. A method for determining at least a portion of a sequence of at least one target template nucleic acid molecule, the method comprising
sequencing regions of at least one target template nucleic acid molecule comprising mutations to provide a plurality of mutated sequence reads,
carrying out the method of any preceding claim on the obtained plurality of mutated sequence reads.

28. The method of claim 27, wherein the step of sequencing comprises
(a) providing a pair of samples, each sample comprising at least one target template nucleic acid molecule;
(b) sequencing regions of the at least one target template nucleic acid molecule in a first of the pair of samples to provide the plurality of non-mutated sequence reads;
(c) introducing mutations into the at least one target template nucleic acid molecule in a second of the pair of samples to provide at least one mutated target template nucleic acid molecule;
(d) sequencing regions of the at least one mutated target template nucleic acid molecule to provide the plurality of mutated sequence reads.

29. The method of claim 28, wherein the step of introducing mutations comprises introducing transition mutations into the at least one target template nucleic acid molecule in the second of the pair of samples.


SEQUENCE LISTING (2 sequences)

SEQ ID NO: 1 - DNA, 45 bp, Artificial Sequence
  Exemplary mutated sequence read
acggaaagcgctacgagcgactgatattgagctacgttcagagcc

SEQ ID NO: 2 - DNA, 28 bp, Artificial Sequence
  An example mutated sequence read
acgcaaagcgctacgagcgactgatatt